(19) United States
(12) Patent Application Publication
(10) Pub. No.: US 2008/0188513 A1
(43) Pub. Date: Aug. 7, 2008

(54) 1-(2-Methylpropyl)-1H-Imidazo[4,5-C](1,5]Naphthyridin-4-Amine Ethanesulfonate and 1-(2-Methylpropyl)-1H-Imidazo[4,5-C](1,5]Naphthyridin-4-Amine Methanesulfonate

(75) Inventors: Raymond D. Skwierczynski, Cambridge, MA (US); Myles L. Brostrom, Saint Paul, MN (US); Tai T. Tran, Saint Paul, MN (US); Joel R. Jacobson, Saint Paul, MN (US)

Correspondence Address: FOLEY AND LARDNER LLP;SUITE 500, 3000 K STREET NW, WASHINGTON, DC, 20007, US

(73) Assignee: taked Pharmaceutical Company Limited

(21) Appl. No.: 11/813,005

(22) PCT Filed: Dec. 28, 2005
(86) PCT No.: PCT/US2005/047067

§ 371 Date: Oct. 1, 2007

Related U.S. Application Data
(60) Provisional application No. 60/640,490, filed on Dec. 30, 2004.
(60) Provisional application No. 60/649,932, filed on Feb. 4, 2005.
(60) Provisional application No. 60/698,416, filed on Jul. 12, 2005.
(60) Provisional application No. 60/708,636, filed on Aug. 16, 2005.

(51) Int. Cl. A61K 31/4375; C07D 471/14; A61P 31/12

(52) U.S. Cl. 514/293; 546/82

(57) ABSTRACT

This invention provides methanesulfonate and ethanesulfonate salts of 1-(2-methylpropyl)-1H-imidazo[4,5-c][1,5]naphthyridin-4-amine, pharmaceutical compositions containing the salts, methods of making, and methods of use.

### CROSS-REFERENCE TO RELATED APPLICATIONS

The present application claims priority to U.S. Provisional Application 60/640,490, filed on Dec. 30, 2004, to U.S. Provisional Application 60/708,636, filed on Aug. 16, 2005, to U.S. Provisional Application 60/649,932, filed on Feb. 4, 2005, and to U.S. Provisional Application 60/698,416 filed on Jul. 12, 2005, all of which are incorporated by reference herein in their entirety.

### BACKGROUND

The compound 1-(2-methylpropyl)-1H-imidazo[4,5-c][1,5]naphthyridin-4-amine has been found to be a useful immune response modifier (IRM) due to its ability to induce cytokine biosynthesis (U.S. Pat. No. 6,194,425). However, formulating and manufacturing pharmaceutical products can present many unforeseen challenges.

### SUMMARY OF THE INVENTION

It has now been found that 1-(2-methylpropyl)-1H-imidazo[4,5-c][1,5]naphthyridin-4-amine and certain pharmaceutically acceptable salts thereof can be very difficult to formulate because of, among other things, particularly low aqueous solubility. For example, even the hydrochloride salt of 1-(2-methylpropyl)-1H-imidazo[4,5-c][1,5]naphthyridin-4-amine has low aqueous solubility although hydrochloride salts of drug substances are commonly made and often soluble. It has also been found, however, that the ethanesulfonate and methanesulfonate salts of 1-(2-methylpropyl)-1H-imidazo[4,5-c][1,5]naphthyridin-4-amine have surprisingly desirable properties, including both good aqueous solubility and good physical and chemical stability in solid form. These are both beneficial characteristics for formulation and manufacture of a useful product.

In some aspects, the invention thus provides methanesulfonate and ethanesulfonate salts of 1-(2-methylpropyl)-1H-imidazo[4,5-c][1,5]naphthyridin-4-amine.

In one aspect, the invention provides 1-(2-methylpropyl)-1H-imidazo[4,5-c][1,5]naphthyridin-4-amine methanesulfonate salt (I) shown below.

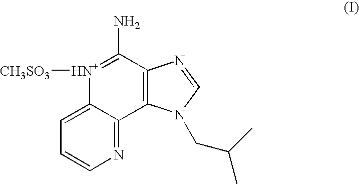

In another aspect, the present invention provides 1-(2-methylpropyl)-1H-imidazo[4,5-c][1,5]naphthyridin-4-amine ethanesulfonate salt (II) shown below.

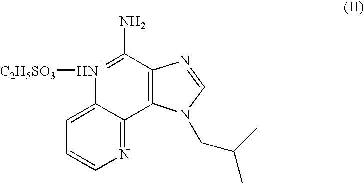

Salt I and salt II may be in solvate or hydrate form, which can provide improved stability during manufacture.

In some embodiments, the invention further provides pharmaceutical compositions that include salt I or II, or a solvate or hydrate thereof, or combinations thereof. Preferably, the pharmaceutical compositions include a pharmaceutically acceptable carrier and an effective amount of salt I or II, or a solvate or hydrate thereof, or combinations thereof. The pharmaceutical compositions will generally have the salt of formula I or II present in dissolved form.

In some embodiments, the invention also provides pharmaceutical compositions prepared by a method that includes combining a pharmaceutically acceptable carrier and an effective amount of salt I or II, or a solvate or hydrate thereof, or combinations thereof.

In some embodiments, the present invention also provides methods of use, for example, methods of inducing cytokine biosynthesis in an animal, treating a viral disease in an animal, and/or treating a neoplastic disease in an animal by administering an effective amount of salt I or II, or a solvate or hydrate thereof, or combinations thereof, optionally in a pharmaceutical composition, to the animal. In one embodiment, the invention further provides a method of treating high risk cervical HPV infection by applying to the cervix a topical formulation comprising dissolved salt 1-(2-methylpropyl)-1H-imidazo[4,5-c][1,5]naphthyridin-4-amine methanesulfonate or 1-(2-methylpropyl)-1H-imidazo[4,5-c][1,5]naphthyridin-4-amine ethanesulfonate.

In another embodiment, the invention provides a method for preparing 1-(2-methylpropyl)-1H-imidazo[4,5-c][1,5]naphthyridin-4-amine ethanesulfonate. The method includes combining 1-(2-methylpropyl)-1H-imidazo[4,5-c][1,5]naphthyridin-4-amine free base with ethanesulfonic acid and a carrier to form a mixture, wherein the carrier includes an organic liquid and optionally water; and allowing the components of the mixture to react under sufficient conditions to form 1-(2-methylpropyl)-1H-imidazo[4,5-c][1,5]naphthyridin-4-amine ethanesulfonate.

In another embodiment, the invention provides a method for preparing 1-(2-methylpropyl)-1H-imidazo[4,5-c][1,5]naphthyridin-4-amine methanesulfonate. The method includes combining 1-(2-methylpropyl)-1H-imidazo[4,5-c][1,5]naphthyridin-4-amine free base with methanesulfonic acid and a carrier to form a mixture, wherein the carrier includes an organic liquid and optionally water; and allowing the components of the mixture to react under sufficient conditions to form 1-(2-methylpropyl)-1H-imidazo[4,5-c][1,5]naphthyridin-4-amine methanesulfonate.

The terms “comprising” and variations thereof do not have a limiting meaning where these terms appear in the description and claims.

As used herein, “a”, “an”, “the”, “at least one”, “at least a portion of” and “one or more” are used interchangeably. Thus, for example, a carrier that comprises an organic liquid can be interpreted to mean that the carrier includes “one or more” organic liquids.

The term “effective amount” (or “therapeutically effective amount”) means an amount of the salt sufficient to induce a therapeutic or prophylactic effect, such as cytokine induction, antitumor activity, and/or antiviral activity. The exact amount of salt used in a pharmaceutical composition of the invention will vary according to factors known to those of skill in the art, such as the physical and chemical nature of the compound, the nature of the carrier, and the intended dosing regimen.

The term “solvate” refers to an amorphous or crystalline material (preferably, crystalline material) having one or more molecules of associated solvent (preferably, within the crystal lattice). The term “hydrate” refers to a solvate wherein the associated molecules are water. A monohydrate has one molecule of water per molecule of the IRM. The water content refers to wt-% water as determined by the known Karl Fisher method.

Certain compounds can crystallize in more than one type of molecular packing with more than one type of internal crystal lattice. The respective resulting crystal structures can have, for example, different unit cells. This phenomenon of “identical chemical structure but different internal structure” is referred to as polymorphism, and the species having different molecular structures are referred to as polymorphs.

As used herein, the term “polymorph” includes both true polymorphs, which have identical chemical structure, and pseudopolymorphs, which contain different hydration and/or solvent levels in the unit cell.

As used herein in connection with a measured quantity, the term “about” refers to that variation in the measured quantity as would be expected by the skilled artisan making the measurement and exercising a level of care commensurate with the objective of the measurement and the precision of the measuring equipment used.

As used herein in connection with a spectrum (e.g., NMR, IR) or an X-ray diffraction pattern shown in a figure, the term “substantially” refers to the fact that the peak positions can shift up to the errors provided and peak intensities can vary as would be expected by one of skill in the art, depending on sample preparation and experimental technique.

The salts 1-(2-methylpropyl)-1H-imidazo[4,5-c][1,5]naphthyridin-4-amine ethanesulfonate and 1-(2-methylpropyl)-1H-imidazo[4,5-c][1,5]naphthyridin-4-amine methanesulfonate described herein may be in any of their pharmaceutically acceptable forms, including solvates, hydrates, polymorphs, and the like, as well as dissolved. It should be understood that the term “salt” includes any or all of such forms, whether explicitly stated or not (although at times, “solvates” and “hydrates” are explicitly stated). The salts described herein may be amorphous or crystalline solids, with or without any associated solvent or water molecules, and may be wholly or partially dissolved (e.g. in a pharmaceutical composition). Thus, “salt” can be used to encompass amorphous salt, crystalline salt, crystalline salt hydrate, crystalline salt solvate, salt in solution, and combinations thereof. For example, in the context of the phrase “1-(2-methylpropyl)-1H-imidazo[4,5-c][1,5]naphthyridin-4-amine ethanesulfonate salt,” unless stated specifically the salt may be crystalline, amorphous, hydrated, solvated, or wholly or partially dissolved. In topical pharmaceutical compositions it will often be in dissolved form. Also, in the context of the phrase “1-(2-methylpropyl)-1H-imidazo[4,5-c][1,5]naphthyridin-4-amine ethanesulfonate salt, or a solvate or hydrate thereof,” 1-(2-methylpropyl)-1H-imidazo[4,5-c][1,5]naphthyridin-4-amine ethanesulfonate may be crystalline, amorphous, hydrated, solvated, or wholly or partially dissolved.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive list.

### BRIEF DESCRIPTION OF THE DRAWINGS

FIG. 1 is a representative X-ray diffraction pattern of a crystalline form of 1-(2-methylpropyl)-1H-imidazo[4,5-c][1,5]naphthyridin-4-amine methanesulfonate monohydrate.

FIG. 2 is a representative X-ray diffraction pattern of a crystalline form of 1-(2-methylpropyl)-1H-imidazo[4,5-c][1,5]naphthyridin-4-amine ethanesulfonate monohydrate.

FIG. 3 is a representative solid state ¹³C NMR spectrum of a crystalline form of 1-(2-methylpropyl)-1H-imidazo[4,5-c][1,5]naphthyridin-4-amine ethanesulfonate monohydrate.

FIG. 4 is a representative IR spectrum of a crystalline form of 1-(2-methylpropyl)-1H-imidazo[4,5-c][1,5]naphthyridin-4-amine ethanesulfonate monohydrate.

FIG. 5 is a pH vs. solubility profile for 1-(2-methylpropyl)-1H-imidazo[4,5-c][1,5]naphthyridin-4-amine, which was determined by potentiometric titration.

FIG. 6 is a representative water sorption isotherm curve for a crystalline form of 1-(2-methylpropyl)-1H-imidazo[4,5-c][1,5]naphthyridin-4-amine ethanesulfonate monohydrate.

FIG. 7 is a representative thermogram of a crystalline form of 1-(2-methylpropyl)-1H-imidazo[4,5-c][1,5]naphthyridin-4-amine ethanesulfonate monohydrate, which shows an overlay of data obtained by DSC and TGA.

FIG. 8 is a representative water sorption isotherm curve for a crystalline form of 1-(2-methylpropyl)-1H-imidazo[4,5-c][1,5]naphthyridin-4-amine methanesulfonate monohydrate.

FIG. 9 is a representative thermogram of a crystalline form of 1-(2-methylpropyl)-1H-imidazo[4,5-c][1,5]naphthyridin-4-amine methanesulfonate monohydrate, which shows an overlay of data obtained by DSC and TGA.

### DETAILED DESCRIPTION OF ILLUSTRATIVE EMBODIMENTS

In some aspects, the present invention provides 1-(2-methylpropyl)-1H-imidazo[4,5-c][1,5]naphthyridin-4-amine methanesulfonate salt (I), or a solvate or hydrate thereof, as well as 1-(2-methylpropyl)-1H-imidazo[4,5-c][1,5]naphthyridin-4-amine ethanesulfonate salt (II), or a solvate or hydrate thereof. Such compounds can be in crystalline form. Generally, these salts have sufficient aqueous solubilities to render them useful for water-based formulations. In fact, they have surprisingly better aqueous solubilities (e.g., greater than 10×, and even greater than 25×) than other salts of the same compound, such as the hydrochloride salt.

Various techniques can be used to characterize crystalline material, including, for example, X-ray powder diffraction (XRPD), solid state ¹³C nuclear magnetic resonance (NMR) spectroscopy, solid state infrared (IR) spectroscopy, thermal analysis (e.g., thermogravimetric analysis (TGA), differential thermal analysis (DTA), and differential scanning calorimetry (DSC)), and the like. Typically, any polymorphs presented herein can be characterized to particular advantage using such techniques. For example, different polymorphs of the same compound typically exhibit diffraction patterns with unique sets of diffraction peaks that can be expressed in two-theta angles, and will typically have a unit cell with different interplanar spacings (Angstroms). Such techniques are well known to one of skill in the art. Data presented herein were obtained under the conditions described in the Examples Section.

In one embodiment, the present invention provides 1-(2-methylpropyl)-1H-imidazo[4,5-c][1,5]naphthyridin-4-amine methanesulfonate salt (I), or a solvate or hydrate thereof (i.e., of the salt).

In one embodiment, the 1-(2-methylpropyl)-1H-imidazo[4,5-c][1,5]naphthyridin-4-amine methanesulfonate salt is in dissolved form.

In one embodiment, the 1-(2-methylpropyl)-1H-imidazo[4,5-c][1,5]naphthyridin-4-amine methanesulfonate salt is in solid form.

In one embodiment, the 1-(2-methylpropyl)-1H-imidazo[4,5-c][1,5]naphthyridin-4-amine methanesulfonate salt is in amorphous form.

In one embodiment, the 1-(2-methylpropyl)-1H-imidazo[4,5-c][1,5]naphthyridin-4-amine methanesulfonate salt, or the solvate or hydrate thereof (i.e., of the salt), is in crystalline form.

In one embodiment, the 1-(2-methylpropyl)-1H-imidazo[4,5-c][1,5]naphthyridin-4-amine methanesulfonate salt is in solvated form.

In one embodiment, the 1-(2-methylpropyl)-1H-imidazo[4,5-c][1,5]naphthyridin-4-amine methanesulfonate salt is in hydrate form.

In one embodiment, the 1-(2-methylpropyl)-1H-imidazo[4,5-c][1,5]naphthyridin-4-amine methanesulfonate salt is in the form of a monohydrate.

In one embodiment, the crystalline form of 1-(2-methylpropyl)-1H-imidazo[4,5-c][1,5]naphthyridin-4-amine methanesulfonate (I) monohydrate can be characterized, for example, by X-ray powder diffraction pattern peaks at 8.51 degrees two-theta, 14.12 degrees two-theta, 16.80 degrees two-theta, 17.88 degrees two-theta, 21.43 degrees two-theta, 23.24 degrees two-theta, and 29.16 degrees two-theta, wherein each of these values is ±0.15 degree two-theta.

Alternatively, and preferably, the crystalline monohydrate of (I) can be characterized, for example, by X-ray powder diffraction pattern peaks at 7.15 degrees two-theta, 8.51 degrees two-theta, 14.12 degrees two-theta, 16.80 degrees two-theta, 17.88 degrees two-theta, 18.49 degrees two-theta, 18.88 degrees two-theta, 21.04 degrees two-theta, 21.43 degrees two-theta, 23.24 degrees two-theta, 25.40 degrees two-theta, 27.92 degrees two-theta, 28.77 degrees two-theta, and 29.16 degrees two-theta, wherein each of these values is +0.15 degree two-theta. The crystalline monohydrate of (I) can also be characterized, for example, by relative intensity peak strength levels for the above-identified peaks of medium (7.15 degrees two-theta), medium (8.51 degrees two-theta), medium (14.12 degrees two-theta), medium high (16.80 degrees two-theta), medium (17.88 degrees two-theta), medium (18.49 degrees two-theta), medium (18.88 degrees two-theta), medium high (21.04 degrees two-theta), medium high (21.43 degrees two-theta), high (23.24 degrees two-theta), medium high (25.40 degrees two-theta), medium (27.92 degrees two-theta), medium (28.77 degrees two-theta), and medium high (29.16 degrees two-theta), respectively, wherein peak strengths categorize relative intensities according to the following scheme: High is 85.0-100.0%; Medium High is 70.0%-84.9%; Medium is 20.0%-69.9%; Medium Low is 5.0%-19.9%; and Low is less than 5.0%.

Alternatively, and more preferably, the crystalline monohydrate of (I) can be characterized, for example, by X-ray powder diffraction pattern peaks at 7.15 degrees two-theta, 7.55 degrees two-theta, 8.51 degrees two-theta, 11.83 degrees two-theta, 13.65 degrees two-theta, 14.12 degrees two-theta, 14.87 degrees two-theta, 16.80 degrees two-theta, 17.88 degrees two-theta, 18.49 degrees two-theta, 18.88 degrees two-theta, 19.92 degrees two-theta, 20.24 degrees two-theta, 21.04 degrees two-theta, 21.43 degrees two-theta, 22.24 degrees two-theta, 23.24 degrees two-theta, 24.64 degrees two-theta, 25.40 degrees two-theta, 25.71 degrees two-theta, 27.20 degrees two-theta, 27.92 degrees two-theta, 28.77 degrees two-theta, 29.16 degrees two-theta, 30.94 degrees two-theta, 31.29 degrees two-theta, 32.76 degrees two-theta, 33.56 degrees two-theta, 34.04 degrees two-theta, 34.88 degrees two-theta, and 35.40 degrees two-theta, wherein each of these values is ±0.15 degree two-theta.

In another embodiment, the crystalline form of 1-(2-methylpropyl)-1H-imidazo[4,5-c][1,5]naphthyridin-4-amine methanesulfonate (I) monohydrate can be characterized, for example, by a unit cell with crystal interplanar spacings of about 10.38 Angstroms, about 6.27 Angstroms, about 5.27 Angstroms, about 4.96 Angstroms, about 4.14 Angstroms, about 3.82 Angstroms, and about 3.06 Angstroms.

Alternatively, and preferably, the crystalline monohydrate of (I) can be characterized, for example, by a unit cell with crystal interplanar spacings of about 12.35 Angstroms, about 10.38 Angstroms, about 6.27 Angstroms, about 5.27 Angstroms, about 4.96 Angstroms, about 4.80 Angstroms, about 4.70 Angstroms, about 4.22 Angstroms, about 4.14 Angstroms, about 3.82 Angstroms, about 3.50 Angstroms, about 3.19 Angstroms, about 3.10 Angstroms, and about 3.06 Angstroms.

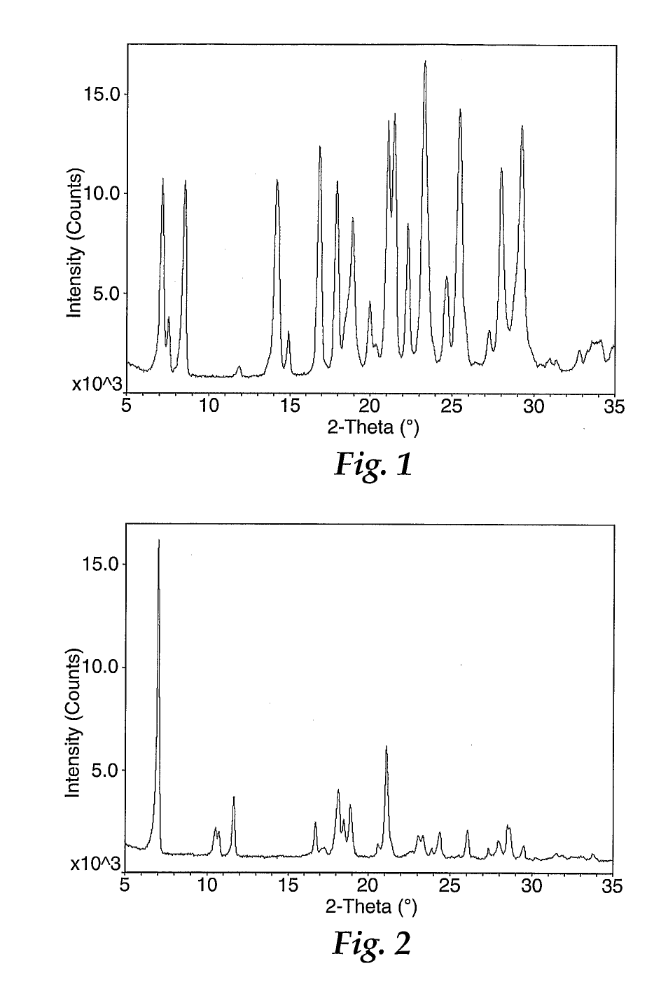

In a particularly preferred embodiment, 1-(2-methylpropyl)-1H-imidazo[4,5-c][1,5]naphthyridin-4-amine methanesulfonate monohydrate can be characterized by an X-ray powder diffraction pattern substantially as depicted in FIG. 1.

In another embodiment, the crystalline form of 1-(2-methylpropyl)-1H-imidazo[4,5-c][1,5]naphthyridin-4-amine methanesulfonate (I) monohydrate can be characterized, for example, by a weight loss of 4.5% to 5.5% over a temperature range of 60° C. to 80° C. as measured by thermogravimetric analysis. Typically, and preferably, this information is coupled with X-ray powder diffraction data, such as, for example, peaks at 7.15 degrees two-theta, 8.51 degrees two-theta, 14.12 degrees two-theta, 16.80 degrees two-theta, 17.88 degrees two-theta, 18.49 degrees two-theta, 18.88 degrees two-theta, 21.04 degrees two-theta, 21.43 degrees two-theta, 23.24 degrees two-theta, 25.40 degrees two-theta, 27.92 degrees two-theta, 28.77 degrees two-theta, and 29.16 degrees two-theta, wherein each of these values is ±0.15 degree two-theta.

In one aspect, the invention also provides a method of preparation of 1-(2-methylpropyl)-1H-imidazo[4,5-c][1,5]naphthyridin-4-amine methanesulfonate (I). The method includes combining the free base of 1-(2-methylpropyl)-1H-imidazo[4,5-c][1,5]naphthyridin-4-amine with methanesulfonic acid and a carrier to form a mixture, and allowing the components of the mixture to react under sufficient conditions to form 1-(2-methylpropyl)-1H-imidazo[4,5-c][1,5]naphthyridin-4-amine methanesulfonate.

The carrier includes an organic liquid and optionally water. In some embodiments, the carrier includes 1 volume percent (vol-% or % v/v) to 15 vol-% water in the organic liquid. In some embodiments, the carrier comprises at least two moles of water per mole of 1-(2-methylpropyl)-1H-imidazo[4,5-c][1,5]naphthyridin-4-amine free base present. Examples of suitable organic liquids include isopropanol (i.e., isopropyl alcohol), other lower alcohols (e.g., methanol, ethanol, n-propanol, n-butanol, sec-butanol), toluene, acetone, acetonitrile, methyl acetate, ethyl acetate, isopropyl acetate, isobutyl acetate, and tetrahydrofuran (THF). Other examples of suitable organic liquids include heptane, tert-butyl methyl ether, N,N-dimethylformamide (DMF), 1-methyl-2-pyrrolidinone (NMP), dichloromethane, and xylene. Mixtures including any two or more of these organic liquids may also be used.

In some embodiments, the method further includes heating the free base, methanesulfonic acid, and/or the carrier prior to combining them, and/or heating the mixture thereof. In certain embodiments, the method further includes forming a precipitate of the 1-(2-methylpropyl)-1H-imidazo[4,5-c][1,5]naphthyridin-4-amine methanesulfonate in the mixture. In one embodiment, forming a precipitate involves cooling the mixture to form a precipitate.

In some embodiments, the method further includes: optionally adding an additional organic liquid to the mixture that includes the precipitate; separating at least a portion of the precipitate from at least a portion of the mixture; washing the precipitate; and at least partially drying the precipitate. Suitable additional organic liquids include ethers (e.g., tert-butyl methyl ether), acetone, THF, 1,2-dimethoxyethane, diethoxymethane, methyl acetate, ethyl acetate, isopropyl acetate, isobutyl acetate, heptanes, toluene, and xylenes.

One skilled in the art will appreciate that there are many ways to separate the precipitate from the mixture, such as filtering, decanting, and centrifugation. In one embodiment, the precipitate is separated by filtration. After separation, the precipitate may optionally be washed. Typically, washing can be carried out with one or more organic liquids (e.g., a lower alcohol, toluene, acetone, acetonitrile, methyl acetate, ethyl acetate, isopropyl acetate, isobutyl acetate, 1,2-dimethoxyethane, diethoxymethane, heptanes, xylenes, and THF) either sequentially or in admixture, to remove impurities. One skilled in the art may appreciate that many methods exist for drying a compound, including for example, using elevated temperatures, desiccation, reduced pressure, or the like.

In one embodiment, the invention provides a pharmaceutical composition that includes a pharmaceutically acceptable carrier and an effective amount of 1-(2-methylpropyl)-1H-imidazo[4,5-c][1,5]naphthyridin-4-amine methanesulfonate (I), or a solvate or hydrate thereof. In another embodiment, the invention provides a pharmaceutical composition prepared by a method that includes combining a pharmaceutically acceptable carrier and an effective amount of (I), or a solvate or hydrate thereof. In one embodiment, the pharmaceutically acceptable carrier includes water.

Further, in certain embodiments, there is provided a method of inducing cytokine biosynthesis in an animal. The method includes administering an effective amount of (I), or a solvate or hydrate thereof, or a pharmaceutical composition containing an effective amount of (I) or a solvate or hydrate thereof, to the animal. In another embodiment, there is provided a method of treating a viral disease in an animal. The method includes administering a therapeutically effective amount of (I), or a solvate or hydrate thereof, or a pharmaceutical composition containing a therapeutically effective amount of (I) or a solvate or hydrate thereof, to the animal. In certain embodiments, the viral disease comprises human papilloma virus located in the cervix. In another embodiment, there is provided a method of treating a neoplastic disease in an animal. The method includes administering a therapeutically effective amount of (I), or a solvate or hydrate thereof, or a pharmaceutical composition containing a therapeutically effective amount of (I) or a solvate or hydrate thereof, to the animal. In certain embodiments, the neoplastic disease is located in the cervix.

In one embodiment, the present invention provides 1-(2-methylpropyl)-1H-imidazo[4,5-c][1,5]naphthyridin-4-amine ethanesulfonate salt (II), or a solvate or hydrate thereof (i.e., of the salt).

In one embodiment, the 1-(2-methylpropyl)-1H-imidazo[4,5-c][1,5]naphthyridin-4-amine ethanesulfonate salt is in dissolved form.

In one embodiment, the 1-(2-methylpropyl)-1H-imidazo[4,5-c][1,5]naphthyridin-4-amine ethanesulfonate salt is in solid form.

In one embodiment, the 1-(2-methylpropyl)-1H-imidazo[4,5-c][1,5]naphthyridin-4-amine ethanesulfonate salt is in amorphous form.

In one embodiment, the 1-(2-methylpropyl)-1H-imidazo[4,5-c][1,5]naphthyridin-4-amine ethanesulfonate salt, or the solvate or hydrate thereof (i.e., of the salt), is in crystalline form.

In one embodiment, the 1-(2-methylpropyl)-1H-imidazo[4,5-c][1,5]naphthyridin-4-amine ethanesulfonate salt is in solvated form.

In one embodiment, the 1-(2-methylpropyl)-1H-imidazo[4,5-c][1,5]naphthyridin-4-amine ethanesulfonate salt is in hydrate form.

In one embodiment, the 1-(2-methylpropyl)-1H-imidazo[4,5-c][1,5]naphthyridin-4-amine ethanesulfonate salt is in the form of a monohydrate.

In one embodiment, the crystalline form of 1-(2-methylpropyl)-1H-imidazo[4,5-c][1,5]naphthyridin-4-amine ethanesulfonate (II) monohydrate can be characterized, for example, by X-ray powder diffraction pattern peaks at 6.98 degrees two-theta, 10.50 degrees two-theta, and 16.70 degrees two-theta, wherein each of these values is ±0.15 degree two-theta.

Alternatively, and preferably, the crystalline monohydrate of (II) can be characterized, for example, by X-ray powder diffraction pattern peaks at 6.98 degrees two-theta, 10.50 degrees two-theta, 16.70 degrees two-theta, 18.11 degrees two-theta, and 26.02 degrees two-theta, wherein each of these values is ±0.15 degree two-theta.

Alternatively, and more preferably, the crystalline monohydrate of (II) can be characterized, for example, by X-ray powder diffraction pattern peaks at 6.98 degrees two-theta, 10.50 degrees two-theta, 10.70 degrees two-theta, 16.70 degrees two-theta, 18.11 degrees two-theta, 18.88 degrees two-theta, 21.11 degrees two-theta, 26.02 degrees two-theta, and 28.51 degrees two-theta, wherein each of these values is ±0.15 degree two-theta. The crystalline monohydrate of (II) can also be characterized, for example, by relative intensity peak strength levels for the above-identified peaks of high (6.98 degrees two-theta), medium low (10.50 degrees two-theta), medium low (10.70 degrees two-theta), medium low (16.70 degrees two-theta), medium (18.11 degrees two-theta), medium (18.88 degrees two-theta), medium (21.11 degrees two-theta), medium low (26.02 degrees two-theta), and medium low (28.51 degrees two-theta), wherein peak strengths categorize relative intensities according to the following scheme: High is 85.0%-100.0%; Medium High is 70.0%-84.9%; Medium is 20.0%-69.9%; Medium Low is 5.0%-19.9%; and Low is less than 5.0%.

Alternatively, and even more preferably, the crystalline monohydrate of (II) can be characterized, for example, by X-ray powder diffraction pattern peaks at 6.98 degrees two-theta, 8.96 degrees two-theta, 10.50 degrees two-theta, 10.70 degrees two-theta, 11.60 degrees two-theta, 14.46 degrees two-theta, 16.70 degrees two-theta, 17.27 degrees two-theta, 18.11 degrees two-theta, 18.47 degrees two-theta, 18.88 degrees two-theta, 20.57 degrees two-theta, 21.11 degrees two-theta, 21.39 degrees two-theta, 22.52 degrees two-theta, 23.04 degrees two-theta, 23.35 degrees two-theta, 23.84 degrees two-theta, 24.35 degrees two-theta, 26.02 degrees two-theta, 27.33 degrees two-theta, 27.92 degrees two-theta, 28.51 degrees two-theta, 29.42 degrees two-theta, 30.19 degrees two-theta, 31.47 degrees two-theta, 31.80 degrees two-theta, 32.45 degrees two-theta, 33.02 degrees two-theta, and 33.73 degrees two-theta, wherein each of these values is ±0.15 degree two-theta.

In another embodiment, the crystalline form of 1-(2-methylpropyl)-1H-imidazo[4,5-c][1,5]naphthyridin-4-amine ethanesulfonate (II) monohydrate can be characterized, for example, by a unit cell with crystal interplanar spacings of about 12.66 Angstroms, about 8.42 Angstroms, and about 5.30 Angstroms.

Alternatively, and preferably, the crystalline monohydrate of (II) can be characterized, for example, by a unit cell with crystal interplanar spacings of about 12.66 Angstroms, about 8.42 Angstroms, about 8.26 Angstroms, about 5.30 Angstroms, about 4.89 Angstroms, about 4.70 Angstroms, about 4.21 Angstroms, about 3.42 Angstroms, and about 3.13 Angstroms.

In a particularly preferred embodiment, 1-(2-methylpropyl)-1H-imidazo[4,5-c][1,5]naphthyridin-4-amine ethanesulfonate (II) monohydrate can be characterized by an X-ray powder diffraction pattern substantially as depicted in FIG. 2.

In one embodiment, 1-(2-methylpropyl)-1H-imidazo[4,5-c][1,5]naphthyridin-4-amine ethanesulfonate monohydrate can be characterized by a ¹³C NMR spectrum having peaks at 127.5 parts per million (ppm), 126.0 ppm, and 122.9 ppm, wherein each of these values is ±0.3 ppm. Alternatively, and preferably, the crystalline monohydrate of (II) can be characterized, for example, by a solid state ¹³C NMR spectrum having peaks at 131.9 ppm, 127.5 ppm, 126.0 ppm, 122.9 ppm, and 56.2 ppm, wherein each of these values is ±0.3 ppm. Alternatively, and more preferably, the crystalline monohydrate of (II) can be characterized, for example, by a solid state ¹³C NMR spectrum having peaks at 148.0 ppm, 134.2 ppm, 131.9 ppm, 127.5 ppm, 126.0 ppm, 122.9 ppm, 56.2 ppm, 45.5 ppm, 29.7 ppm, 21.8 ppm, and 10.5 ppm, wherein each of these values is ±0.3 ppm.

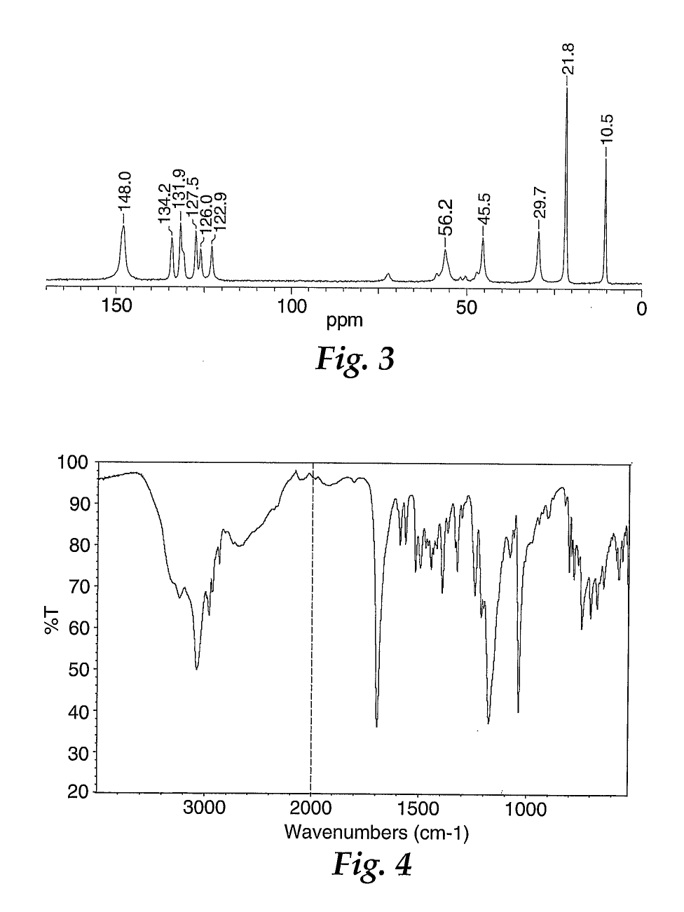

In a particularly preferred embodiment, 1-(2-methylpropyl)-1H-imidazo[4,5-c][1,5]naphthyridin-4-amine ethanesulfonate (II) monohydrate can be characterized by a solid state ¹³C NMR spectrum substantially as depicted in FIG. 3.

In another embodiment, the crystalline monohydrate of 1-(2-methylpropyl)-1H-imidazo[4,5-c][1,5]naphthyridin-4-amine ethanesulfonate (II) can be characterized, for example, by a weight loss of 4.2% to 5.2% over a temperature range of 55° C. to 110° C., preferably over a temperature range of 65° C. to 100° C., as measured by TGA. Alternatively, or additionally, solid state FTIR spectroscopy can be carried out, which can provide characterizing information. For example, the crystalline monohydrate (II) can be characterized by a solid state IR spectrum substantially as depicted in FIG. 4.

Typically, and preferably, the TGA and IR data can be coupled with X-ray powder diffraction data, for example. For example, the crystalline monohydrate of (II) can be characterized by peaks at: 6.98 degree two-theta, 10.50 degree two-theta, 10.70 degree two-theta, 16.70 degree two-theta, 18.11 degree two-theta, 18.88 degree two-theta, 21.11 degree two-theta, 26.02 degree two-theta, and 28.51 degree two-theta, wherein each of these values is +0.15 degree two-theta. Instead of the X-ray diffraction data, solid state ¹³C NMR could be used for characterization along with TGA and IR data. For example, the crystalline monohydrate of (II) can be characterized by peaks at: 148.0 ppm, 134.2 ppm, 131.9 ppm, 127.5 ppm, 126.0 ppm, 122.9 ppm, 56.2 ppm, 45.5 ppm, 29.7 ppm, 21.8 ppm, and 10.5 ppm, wherein each of these values is ±0.3 ppm.

In one aspect, the invention provides a method of preparation of 1-(2-methylpropyl)-1H-imidazo[4,5-c][1,5]naphthyridin-4-amine ethanesulfonate. The method includes combining the free base of 1-(2-methylpropyl)-1H-imidazo[4,5-c][1,5]naphthyridin-4-amine with ethanesulfonic acid and a carrier to form a mixture, and allowing the components of the mixture to react under sufficient conditions to form 1-(2-methylpropyl)-1H-imidazo[4,5-c][1,5]naphthyridin-4-amine ethanesulfonate.

The carrier includes an organic liquid and optionally water. In some embodiments, the carrier includes 1 vol-% to 15 vol-% water in the organic liquid. In some embodiments, the carrier comprises at least two moles of water per mole of 1-(2-methylpropyl)-1H-imidazo[4,5-c][1,5]naphthyridin-4-amine free base present. Examples of suitable organic liquids include isopropanol (i.e., isopropyl alcohol), other lower alcohols (e.g., methanol, ethanol, n-propanol, n-butanol, sec-butanol), toluene, acetone, acetonitrile, methyl acetate, ethyl acetate, isopropyl acetate, isobutyl acetate, and THF. Other examples of suitable organic liquids include heptane, tert-butyl methyl ether, N,N-dimethylformamide (DMF), 1-methyl-2-pyrrolidinone (NMP), dichloromethane, and xylene. Mixtures including any two or more of these organic liquids may also be used.

In some embodiments, the method further includes heating the free base, ethanesulfonic acid, and/or the carrier prior to combining them, and/or heating the mixture thereof. In certain embodiments, the method further includes forming a precipitate of the 1-(2-methylpropyl)-1H-imidazo[4,5-c][1,5]naphthyridin-4-amine ethanesulfonate in the mixture. In one embodiment, forming a precipitate involves cooling the mixture to form a precipitate. Preferably, the cooling occurs at a rate less than 2.0° C. per minute.

In some embodiments, the method further includes: optionally adding an additional organic liquid to the mixture that includes the precipitate; separating at least a portion of the precipitate from at least a portion of the mixture; washing the precipitate; and at least partially drying the precipitate. Suitable additional organic liquids include ethers (e.g., tert-butyl methyl ether), acetone, THF, 1,2-dimethoxyethane, diethoxymethane, methyl acetate, ethyl acetate, isopropyl acetate, isobutyl acetate, heptanes, toluene, and xylenes.

As above, one skilled in the art will appreciate that there are many ways to separate the precipitate from the mixture, such as filtering, decanting, and centrifugation. In one embodiment, the precipitate is separated by filtration. After separation, the precipitate may optionally be washed. Typically, washing can be carried out with one or more organic liquids (e.g., a lower alcohol, toluene, acetone, acetonitrile, methyl acetate, ethyl acetate, isopropyl acetate, isobutyl acetate, 1,2-dimethoxyethane, diethoxymethane, heptanes, xylenes, and THF) either sequentially or in admixture, to remove impurities. One skilled in the art may appreciate that many methods exist for drying a compound, including, for example, using elevated temperatures, desiccation, reduced pressure, or the like.

In one embodiment, the invention provides a pharmaceutical composition that includes a pharmaceutically acceptable carrier and an effective amount of 1-(2-methylpropyl)-1H-imidazo[4,5-c][1,5]naphthyridin-4-amine ethanesulfonate (II), or a solvate or hydrate thereof. In another embodiment, the invention provides a pharmaceutical composition prepared by a method that includes combining a pharmaceutically acceptable carrier and an effective amount of (II), or a solvate or hydrate thereof. In one embodiment, the pharmaceutically acceptable carrier includes water.

Further, in certain embodiments, there is provided a method of inducing cytokine biosynthesis in an animal. The method includes administering an effective amount of (II), or a solvate or hydrate thereof, or a pharmaceutical composition containing an effective amount of (II) or a solvate or hydrate thereof, to the animal. In another embodiment, there is provided a method of treating a viral disease in an animal. The method includes administering a therapeutically effective amount of (II), or a solvate or hydrate thereof, or a pharmaceutical composition containing a therapeutically effective amount of (II) or a solvate or hydrate thereof, to the animal. In certain embodiments, the viral disease comprises human papilloma virus located in the cervix. In another embodiment, there is provided a method of treating a neoplastic disease in an animal. The method includes administering a therapeutically effective amount of (II), or a solvate or hydrate thereof, or a pharmaceutical composition containing a therapeutically effective amount of (II) or a solvate or hydrate thereof, to the animal. In certain embodiments, the neoplastic disease is located in the cervix.

In another embodiment, there is provided a method of treating high risk cervical HPV infection by applying to the cervix a topical formulation comprising dissolved salt 1-(2-methylpropyl)-1H-imidazo[4,5-c][1,5]naphthyridin-4-amine methanesulfonate or 1-(2-methylpropyl)-1H-imidazo[4,5-c][1,5]naphthyridin-4-amine ethanesulfonate.

### Preparation of the Compounds

As used herein, the terms “alkyl” and the prefix “alk-” are inclusive of both straight chain and branched chain groups and of cyclic groups. Unless otherwise specified, these groups contain from 1 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, and cyclohexyl.

Unless otherwise specified “alkylene” is the divalent form of the “alkyl,” defined above. The term “alkylenyl” is used when “alkylene” is substituted. For example, an arylalkylenyl group comprises an alkylene moiety to which an aryl group is attached.

A “lower alcohol” is understood to be a straight chain or branched chain alcohol containing one to four carbon atoms. Examples include, methanol, ethanol, n-propanol, isopropanol, n-butanol, sec-butanol, and tert-butanol.

The term “aryl” in reference to “arylsulfonyl halide” includes carbocyclic aromatic rings or ring systems that may be unsubstituted or substituted. Examples of aryl groups include phenyl, naphthyl, biphenyl, fluorenyl, and indenyl. Examples of substituents that may be present on the aryl group include alkyl, alkoxy, haloalkyl, haloalkoxy, halogen, nitro, hydroxy, cyano, aryl, aryloxy, and arylalkyleneoxy.

1-(2-Methylpropyl)-1H-imidazo[4,5-c][1,5]naphthyridin-4-amine can be prepared according to the route shown in Scheme I. Steps (1) through (6) of reaction Scheme I can be carried out according to the methods described in U.S. Pat. No. 6,194,425 (Gerster et al.) or modifications thereof as described in the EXAMPLES below.

In step (6) of Scheme 1,1-(2-methylpropyl)-5-oxido-1H-imidazo[4,5-c][1,5]naphthyridine is aminated to provide 1-(2-methylpropyl)-1H-imidazo[4,5-c][1,5]naphthyridin-4-amine. Step (6) involves the activation of 1-(2-methylpropyl)-5-oxido-1H-imidazo[4,5-c][1,5]naphthyridine by conversion to an ester and then reacting the ester with an aminating agent. Suitable activating agents include alkyl- or arylsulfonyl chlorides such as benzenesulfonyl chloride, methanesulfonyl chloride, or p-toluenesulfonyl chloride. Suitable aminating agents include ammonia, in the form of ammonium hydroxide, for example, and ammonium salts such as ammonium carbonate, ammonium bicarbonate, and ammonium phosphate. The reaction can be carried out by adding ammonium hydroxide to a solution of 1-(2-methylpropyl)-5-oxido-1H-imidazo[4,5-c][1,5]naphthyridine in a suitable solvent such as dichloromethane or chloroform and then adding p-toluenesulfonyl chloride. The reaction can be carried out at room temperature, and the product can be isolated from the reaction mixture using conventional methods.

Preferably, the reaction in step (6) of reaction Scheme I is carried out by combining ammonium hydroxide, ammonia, or another suitable aminating agent listed above with 1-(2-methylpropyl)-5-oxido-1H-imidazo[4,5-c][1,5]naphthyridine in a suitable medium such as a lower alcohol or a mixture containing a lower alcohol. The resulting mixture can then be combined with an arylsulfonyl halide to provide 1-(2-methylpropyl)-1H-imidazo[4,5-c][1,5]naphthyridin-4-amine in a mixture which is then combined with an aqueous base. The aqueous base is preferably an aqueous alkali metal hydroxide, such as sodium hydroxide or potassium hydroxide. Alternatively, an aqueous solution of potassium carbonate or sodium carbonate may be used. The reaction can be conveniently carried out by adding ammonium hydroxide or an alcoholic ammonia solution to a solution of 1-(2-methylpropyl)-5-oxido-1H-imidazo[4,5-c][1,5]naphthyridine in methanol, ethanol, or isopropanol and then adding benzenesulfonyl chloride or p-toluenesulfonyl chloride. The reaction can be carried out at room temperature. Aqueous sodium hydroxide can then be added, and the product can be isolated using conventional techniques. Typically the product precipitates under the reaction conditions and can be separated from the reaction mixture by conventional methods.

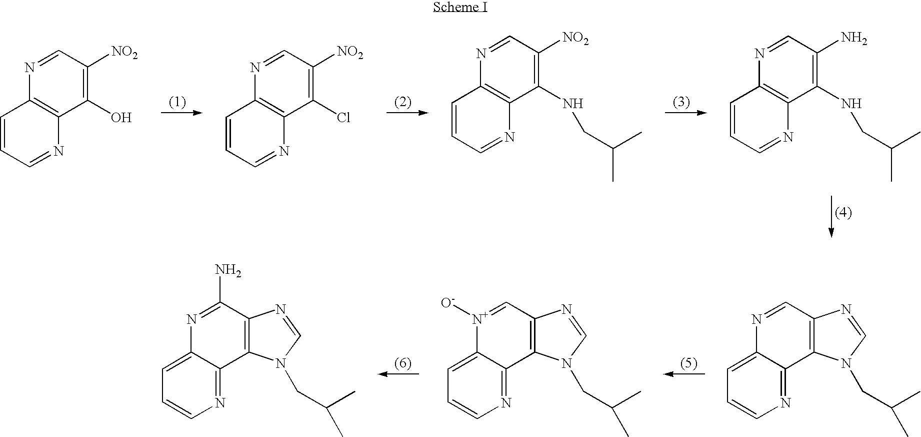

1-(2-Methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate, including a solvate or hydrate thereof, can be prepared by combining 1-(2-methylpropyl)-1H-imidazo[4,5-c][1,5]naphthyridin-4-amine with either ethanesulfonic acid or methanesulfonic acid, respectively, and a carrier, wherein the carrier comprises an organic liquid and optionally water, and allowing the components of the mixture to react under sufficient conditions to form the desired salt. Preferably, and particularly for crystalline solid forms, 1-(2-methylpropyl)-1H-imidazo[4,5-c][1,5]naphthyridin-4-amine ethanesulfonate and 1-(2-methylpropyl)-1H-imidazo[4,5-c][1,5]naphthyridin-4-amine methanesulfonate are typically prepared according to Scheme II. In steps (1) and (2) of Scheme II, a solution or suspension of the free base of 1-(2-methylpropyl)-1H-imidazo[4,5-c][1,5]naphthyridin-4-amine is prepared by combining the free base with one or more organic liquids or a mixture of one or more organic liquids and water. The resulting solution or suspension may be heated to an elevated temperature, for example, the reflux temperature of the solvent, before combining it with ethanesulfonic acid or methanesulfonic acid in step (3). The solution or suspension may also be combined with the acid at room temperature. Solutions of the acid or the neat compound may be used. Several organic liquids may be used; these include, for example, a lower alcohol, toluene, acetone, acetonitrile, methyl acetate, ethyl acetate, isopropyl acetate, isobutyl acetate, and tetrahydrofuran (THF). Other examples of organic liquids that may be used include heptane, tert-butyl methyl ether, N,N-dimethylformamide (DMF), 1-methyl-2-pyrrolidinone (NMP), dichloromethane, and xylene. Mixtures including any two or more of these organic liquids may also be used. Useful mixtures of organic liquids include mixtures of heptane and tert-butyl methyl ether, isopropanol, 2-butanol, THF, dichloromethane, ethyl acetate, or toluene; mixtures of tert-butyl methyl ether and a lower alcohol, THF, dichloromethane, ethyl acetate, or toluene; mixtures of isopropanol and dichloromethane, ethyl acetate, or toluene; mixtures of 2-butanol and THF, dichloromethane, or toluene; mixtures of THF and dichloromethane, ethyl acetate, or toluene; dichloromethane and ethyl acetate; dichloromethane and toluene; and ethyl acetate and toluene. Water can optionally be combined with the organic liquid or organic liquid mixture. Useful amounts of water that may be combined with the organic liquid or organic liquid mixture include 1% to 15% v/v (volume percent). Preferably, the amount of water that is combined is 1% to 10% v/v. More preferably, the amount of water is 1% to 5% v/v, and most preferably 1% to 3% v/v. Many commercially available organic solvents already contain up to 1% v/v water. Preferably, the carrier used in steps (1) through (3) of Scheme II is 2% v/v water in ethyl acetate, 2% v/v water in isopropyl alcohol, or isopropyl alcohol. The amount of water that can be combined with the organic liquid may also be measured in terms of equivalents (mole/mole) with respect to the amount of 1-(2-methylpropyl)-1H-imidazo[4,5-c][1,5]naphthyridin-4-amine free base present. Preferably, the amount of water present is not less than 2 equivalents (mole/mole) with respect to the amount of 1-(2-methylpropyl)-1H-imidazo[4,5-c][1,5]naphthyridin-4-amine. More preferably, 2 to 10 moles of water are present for every mole of 1-(2-methylpropyl)-1H-imidazo[4,5-c][1,5]naphthyridin-4-amine. Most preferably, 2 to 5 moles of water are present for every mole of 1-(2-methylpropyl)-1H-imidazo[4,5-c][1,5]naphthyridin-4-amine. The heating of the solution or suspension prepared in step (3) may be carried out for a few minutes, more than 15 minutes, more than an hour, more than four hours, or more than 24 hours, for example, up to the limit of when the salt begins to decompose.

In steps (4a) and (4b) of Scheme II, the hot solution of the salt is cooled to form a precipitate. The cooling in steps (4a) and (4b) may be carried out to room temperature or below room temperature in the range of −20° C. to 25° C. Steps (4a) and (4b) differ in the rate of cooling that is used. In step (4b), a slow cooling rate is used. A slow rate may be less than 2° C./minute; preferably it is less than 1° C./minute. More preferably, a slow rate is less than 0.75° C./minute. Most preferably, it may be in the range of 0.1° C./minute to 0.5° C./minute. In some cases it may be as low as 0.05° C./minute. In step (4a), a fast cooling rate is used. A fast cooling rate may be characterized by a rate that is achieved when a small volume (less than 100 mL) of solution or suspension is allowed to cool while standing at room temperature. Preferably, a fast cooling rate is greater than 2° C./minute; and more preferably it is greater than 4° C./minute. For the preparation of 1-(2-methylpropyl)-1H-imidazo[4,5-c][1,5]naphthyridin-4-amine ethanesulfonate in 1% to 15% (v/v) water/isopropyl alcohol or in the presence of at least two moles of water for every mole of 1-(2-methylpropyl)-1H-imidazo[4,5-c][1,5]naphthyridin-4-amine present a slow cooling rate is preferred. Preferably, the cooling rate is less than 2° C./minute. More preferably, it is in the range of 0.1° C./minute to 0.5° C./minute.

Other methods of forming a precipitate are possible as would be understood by one skilled in the art. These methods include, for example, introducing another organic liquid in which the salt is poorly soluble. For example, tert-butyl methyl ether can be combined with a solution of the salt in acetonitrile, 2-butanol, dichloromethane, or THF, wherein any of these solutions may contain at least two moles of water for every mole of salt. The salt solution may be added to tert-butyl methyl ether, or tert-butyl methyl ether can be added slowly or quickly to the salt solution. Preferably, tert-butyl methyl ether is added slowly to the salt solution to form a precipitate.

In step (5a) or (5c) of Scheme II, the precipitated salt is separated from the suspension. Usually, this is carried out by filtration, but other methods, such as decanting the liquid, are known to one skilled in the art. In step (5b) of Scheme II, an additional liquid is added to the suspension before the separation of step (6b). The additional liquid may be used to help separate the solid by facilitating the removal of the suspension from the reaction flask. The additional liquid may be an ether, for example, tert-butyl methyl ether. The additional liquid may also be acetone, THF, 1,2-dimethoxyethane, diethoxymethane, methyl acetate, ethyl acetate, isopropyl acetate, isobutyl acetate, heptanes, toluene, and/or xylenes.

In step (6a) or (7) the separated solid is washed and dried. Washing the solid is usually carried out using one or more organic liquids or a mixture of an one or more organic liquids and water. The same liquids described in steps (1) and (2) or step (5b) may be used. Drying in step (6a) or (7) may be carried out by heating, by placing the solid under vacuum, by using a flow of inert gas, or combinations thereof. During the drying step, heating in the range of 25° C. to 65° C. is preferred. When drying is carried out under at least a partial vacuum, a vacuum in the range of 2×10² Pascals (Pa) to 1×10⁵ Pa is preferred. For the preparation of 1-(2-methylpropyl)-1H-imidazo[4,5-c][1,5]naphthyridin-4-amine ethanesulfonate, drying is preferably carried out under a vacuum in the range of 2×10² Pa to 1×10⁵ Pa at a temperature in the range of 35° C. to 55° C., more preferably, 40° C. to 50° C.

Other processes for preparing 1-(2-methylpropyl)-1H-imidazo[4,5-c][1,5]naphthyridin-4-amine ethanesulfonate and 1-(2-methylpropyl)-1H-imidazo[4,5-c][1,5]naphthyridin-4-amine methanesulfonate (including hydrates or solvates thereof), particularly in crystalline form, are described in the EXAMPLES below.

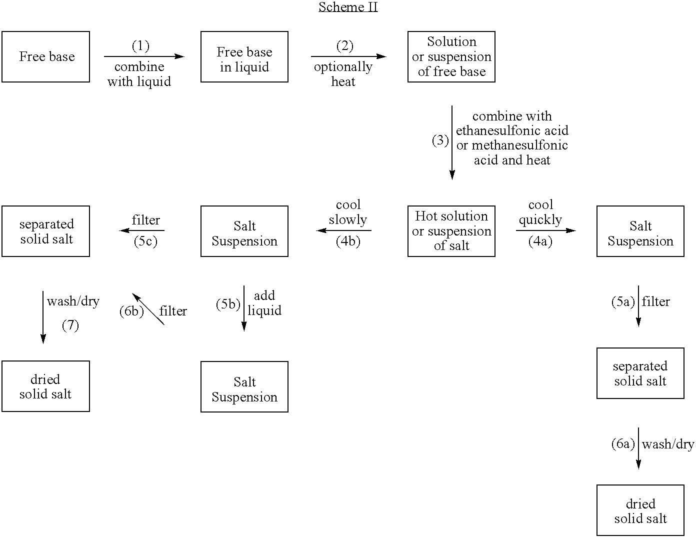

### Pharmaceutical Compositions and Biological Activity

Pharmaceutical compositions of the invention contain a therapeutically effective amount of 1-(2-methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate (including a hydrate or solvate thereof) in combination with a pharmaceutically acceptable carrier. Certain pharmaceutical compositions may contain 1-(2-methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate (including a hydrate or solvate thereof) in crystalline form.

1-(2-Methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate can be administered as the single therapeutic agent in the treatment regimen, or it may be administered in combination with one another or with other active agents, including additional immune response modifiers, antivirals, antibiotics, antibodies, proteins, peptides, oligonucleotides, etc.

Salts (including hydrates and solvates) of the invention have been shown to induce the production of certain cytokines in experiments performed according to the tests set forth below. These results indicate that the salts are useful as immune response modifiers that can modulate the immune response in a number of different ways, rendering them useful in the treatment of a variety of disorders.

Cytokines whose production may be induced by the administration of salts of the invention generally include interferon-α (IFN-α) and/or tumor necrosis factor-α (TNF-α) as well as certain interleukins (IL). Cytokines whose biosynthesis may be induced by salts of the invention include IFN-α, TNF-α, IL-1, IL-6, IL-10 and IL-12, and a variety of other cytokines. Among other effects, these and other cytokines can inhibit virus production and tumor cell growth, making the salts useful in the treatment of viral diseases and neoplastic diseases. Accordingly, the invention provides a method of inducing cytokine biosynthesis in an animal comprising administering an effective amount of a salt or composition of the invention to the animal. The animal to which the salt or composition is administered for induction of cytokine biosynthesis may have a disease as described infra, for example a viral disease or a neoplastic disease, and administration of the salt may provide therapeutic treatment. Alternatively, the salt may be administered to the animal prior to the animal acquiring the disease so that administration of the salt may provide a prophylactic treatment.

In addition to the ability to induce the production of cytokines, salts of the invention can affect other aspects of the innate immune response. For example, natural killer cell activity may be stimulated, an effect that may be due to cytokine induction. The salts may also activate macrophages, which in turn stimulate secretion of nitric oxide and the production of additional cytokines. Further, the salts may cause proliferation and differentiation of B-lymphocytes.

Salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5, and IL-13 may be inhibited upon administration of the salts.

Whether for prophylaxis or therapeutic treatment of a disease, and whether for effecting innate or acquired immunity, the salt or composition may be administered alone or in combination with one or more active components as in, for example, a vaccine adjuvant. When administered with other components, the salt and other component or components may be administered separately; together but independently such as in a solution; or together and associated with one another such as (a) covalently linked or (b) non-covalently associated, e.g., in a colloidal suspension.

Conditions for which IRMs identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such as fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogenous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, an IRM salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens; toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease. The salts of the invention, including topical formulations thereof, may be used as adjuvants in conjunction with intramuscular, intradermal, subcutaneous, mucosal (e.g., nasal or vaginal) or any other vaccination delivery. For example, topical formulations of the salts of the invention may be used for dermal vaccination or mucosal vaccination nasally or vaginally in combination with an HPV vaccine (such as GARDASIL and CERVARIX).

Certain IRM salts of the present invention may be particularly helpful in individuals having compromised immune function. For example, certain salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a salt of the invention to the animal. A particular use of the IRM salts of the invention, 1-(2-methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate, is for treatment of diseases of the cervix, such as HPV infection and/or cervical neoplasias. It is believed that certain so-called high risk HPV subtypes, such has HPV 16, 18, and others are especially pernicious and may lead to cervical cancer. Formulations and such uses of salt compounds of the invention are disclosed in copending U.S. application 60/698,416.

It will also be understood that the salts described herein may be used in combination with any number of other drugs and devices. For example, combinations may be made with any other antiviral or anticancer drug, as are well known in the art, including antibodies, chemotherapeutics, direct antiviral drugs, immunotherapeutics, as well as chemoablatives, laser ablation, cryotherapy, and surgical excision. In treatment of HPV using the salts of the invention it may also be useful to use products to detect the presence or absence of various HPV subtypes (e.g., from Digene Corp.), either to determine whether treatment is necessary or to determine whether treatment has been effective at reducing or eliminating the virus.

An amount of a salt effective to induce cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased over a background level of such cytokines. The precise amount will vary according to factors known in the art but is expected to be a dose of about 100 nanograms per kilograms (ng/kg) to about 50 milligrams per kilogram (mg/kg), preferably about 10 micrograms per kilogram (μg/kg) to about 5 mg/kg. The invention also provides a method of treating a viral infection in an animal and a method of treating a neoplastic disease in an animal comprising administering an effective amount of a salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg.

### Test Methods

### Powder X-ray Diffraction Analysis

Reflection geometry data were collected in the form of a (θ/2θ) survey scan by use of a Philips (PANalytical, Natick, Mass.) vertical diffractometer, copper K_{α} radiation, and proportional detector registry of the scattered radiation. The diffractometer is fitted with variable incident beam slits, fixed diffracted beam slits, and a graphite diffracted beam monochromator. The sample was mulled in an agate mortar and applied as a dry powder to a zero background specimen holder composed of single crystal quartz. The survey scan was conducted from 3 to 55 degrees (2θ) using a 0.04-degree step size and 6-second dwell time. X-ray generator settings of 45 kV and 35 mA were employed. Analysis of resulting powder diffraction data was accomplished by use of Jade (version 6, Materials Data Inc., Livermore, Calif.) diffraction software suite.

### Solid State ¹³C Nuclear Magnetic Resonance (NMR) Spectroscopy

Solid State ¹³C NMR spectra were obtained on a Varian 400 MHz wide-bore INOVA spectrometer using a variable amplitude cross-polarization pulse sequence. The chemical shifts were referenced to tetramethylsilane using hexamethyl benzene as an external standard. Due to the experimental conditions (magic angle alignment and lock signal drift), the chemical shifts may vary in the range of +/−0.3 ppm.

### Thermal Analysis

Differential Scanning Calorimetric (DSC) analysis and Thermogravimetric Analysis (TGA) were performed using TA Instruments MDSC Q1000 and TGA 2950, respectively. The DSC instrument was calibrated using indium and tin standards. The temperature calibration on the TGA instrument was performed using nickel and alumel standards. A heating rate of 10° C./minute and a nitrogen purge at 50 mL/minute were applied for both DSC and TGA. For DSC, a hermetically sealed sample pan with a pinhole was used to reduce the measurement variability of the dehydration event.

### Water Sorption Analysis

The automated analysis of water sorption was performed using a VTI Symmetrical Gravimetric Analyzer Model 100 (SGA-100) operated in Step-Isotherm mode. The sample was exposed to a cycle of relative humidity (RH) conditions at 25° C. The relative humidity was cycled from 5% to 95% and back to 5% in increments of 5%, and the weight of the sample at each stage was recorded after equilibration (less than 0.01% weight change within 5 minutes). The percent weight change of the sample as a function of the relative humidity was plotted to yield the adsorption/desorption isotherm.

### Fourier Transform Infrared Spectroscopy (FTIR)

Infrared spectra were obtained on a Nicolet Magna-IR Spectrometer 750, using an ENDURANCE single-reflection attenuated total reflection (ATR) accessory. A neat sample was placed on the diamond sampling crystal, and the spectrum was collected from 4000 to 525 wavenumbers (cm⁻¹). ATR spectral correction software was utilized.

### Aqueous Solubility of 1-(2-methylpropyl)-1H-imidazo[4,5-c][1,5]naphthyridin-4-amine

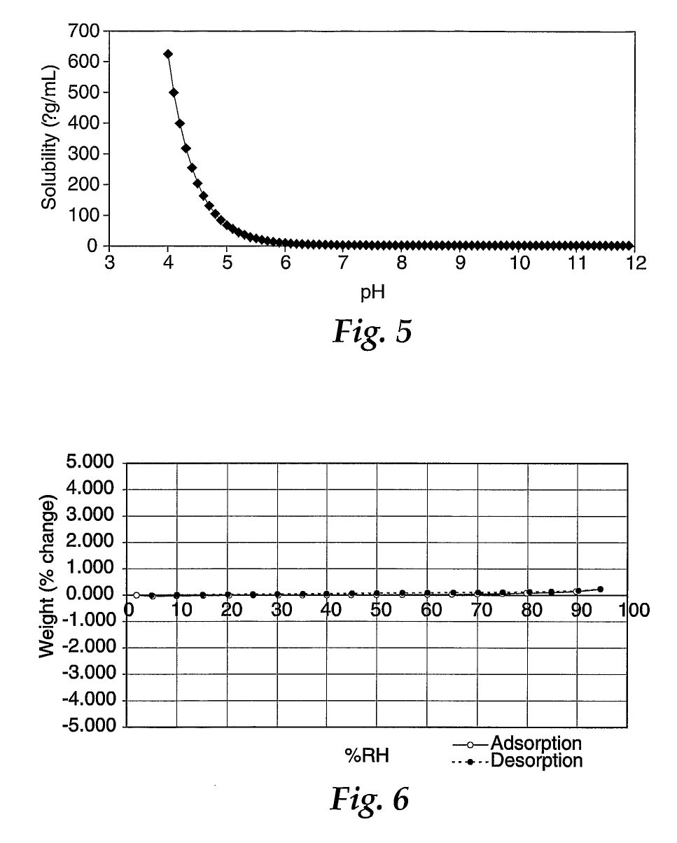

The aqueous solubility of 1-(2-methylpropyl)-1H-imidazo[4,5-c][1,5]naphthyridin-4-amine free base was measured potentiometrically as a function of pH. A p-SOL microtitrator (available from pION, Inc., 5 Constitution Way, Woburn, Mass.) was used to obtain the intrinsic solubility and pH/solubility profile in 0.15 M aqueous potassium chloride. The titration was carried out on a sample approximately 3 mg in size. The intrinsic solubility (Sₒ) determined by this method was 0.003 mg/mL. The pKₐ of 1-(2-methylpropyl)-1H-imidazo[4,5-c][1,5]naphthyridin-4-amine, determined potentiometrically in a separate experiment to be 6.1, was used in the calculation to determine the intrinsic solubility. The curve showing solubility as a function of pH for 1-(2-methylpropyl)-1H-imidazo[4,5-c][1,5]naphthyridin-4-amine free base is shown in FIG. 5.

### EXAMPLES

Objects and advantages of this invention are further illustrated by the following examples, but the particular materials and amounts thereof recited in these examples, as well as other conditions and details, should not be construed to unduly limit this invention.

### Example 1

### Preparation of 1-(2-Methylpropyl)-1H-imidazo[4,5-c][1,5]naphthyridin-4-amine ethanesulfonate monohydrate

### Part A

Under an argon atmosphere, 1,5-naphthyridin-4-ol (1.6 kilograms (kg), 11 moles (mol)) was added in portions of 160 grams (g) with continuous stirring to fuming nitric acid (16 liters (L)) while maintaining the reaction temperature at 45.5° C. or below. After the addition, the reaction was stirred for 23 minutes at about 45° C., heated to reflux over a period of 2.25 hours, heated at reflux (90° C. to 95° C.) for five hours, and allowed to cool to room temperature overnight. The reaction mixture was then cooled to 7.5° C., and water (16 L) was slowly added while maintaining the reaction temperature below 25° C. The resulting mixture was cooled to 9° C., and ammonium hydroxide (20 L) was slowly added to adjust the mixture to pH 6.2 while maintaining the temperature below 15° C. The resulting mixture was stirred for ten minutes and cooled to 2.8° C. The resulting solid was isolated by filtration, washed with cold water (2×2.2 L, 4° C.), dried under vacuum at room temperature, and dried under vacuum at 75° C. for 47 hours to provide 1.778 kg of 3-nitro[1,5]naphthyridin-4-ol.

### Part B

Under an argon atmosphere, a solution of 3-nitro[1,5]naphthyridin-4-ol (1.778 kg, 9.30 mol) in N,N-dimethylformamide (DMF) (16 L) was stirred for 45 minutes at 17° C. Phosphorous oxychloride (2.095 kg, 13.7 mol) was added slowly while maintaining the temperature at about 20° C., and then the reaction was stirred for 15.25 hours at 20° C. With continuous stirring, the reaction mixture was then added over a period of 55 minutes to water (76 L) that had been cooled to 4.5° C. During the addition, the temperature of the mixture was not allowed to exceed 10° C., and the temperature was 9.5° C. at the end of the addition. The mixture resulting from the addition was stirred for 100 minutes while cooling from 9.5° C. to 2.5° C. A solid formed and was isolated by filtration, washed with water (2×8 L), and dried with suction to provide 3.3 kg of 4-chloro-3-nitro[1,5]naphthyridine.

### Part C

A solution of the material from Part B in dichloromethane (26 L) was heated to 31° C., and sodium sulfate (2 kg) and magnesium sulfate (500 g) were added. The resulting mixture was stirred for one hour and then filtered. The filter cake was washed with dichloromethane (5 L), and the filtrate was transferred to another vessel with additional dichloromethane (8 L). Under an argon atmosphere and with continuous stirring, isobutylamine (2.5 L) was added to the filtrate while maintaining a reaction temperature of 17° C. to 24° C. The reaction was stirred for 13.5 hours at a temperature of 17° C. to 24° C. and then concentrated to dryness under reduced pressure at 40° C. The resulting solid was mixed with water (18 L), and the resulting mixture was stirred at 20° C. to 21° C. for three hours and then filtered. The isolated solid was washed with water (3×3 L), pulled dry under vacuum, and further dried under vacuum for 16.5 hours at 75° C. to provide 1.98 kg of N⁴-(2-methylpropyl)-3-nitro[1,5]naphthyridin-4-amine. The product was split into five portions.

### Part D

A Parr vessel was charged with toluene (3.86 L), 2-propanol (386 milliliters (mL)), N⁴-(2-methylpropyl)-3-nitro[1,5]naphthyridin-4-amine (386 g, 1.56 mol), and 5% platinum on carbon (77.2 g, 50% w/w (weight percent) in water). The vessel was sealed and purged three times with nitrogen while the reaction mixture was stirred. The reaction mixture was then placed under hydrogen pressure (2.1×10⁵ Pascals (Pa) to 4.1×10⁵ Pa, 30 pounds per square inch (psi) to 60 psi) for 130 minutes while maintaining the temperature between 18° C. and 22° C. This reaction was repeated four more times with reaction times ranging from 120 minutes to 215 minutes and reaction temperatures ranging from 19° C. to 24° C. The five runs were combined, treated with magnesium sulfate (2 kg), allowed to stand for 90 minutes, and filtered through a layer of CELITE filter agent. The filter cake was washed with 1:1 toluene/2-propanol (4 L) and then toluene (16 L), and the filtrate was concentrated under reduced pressure at approximately 40° C. to provide 1.59 kg of N⁴-(2-methylpropyl)[1,5]naphthyridin-3,4-diamine as an oil.

Parts A through D were repeated on the same scale to provide an additional 1.236 kg of N⁴-(2-methylpropyl)[1,5]naphthyridin-3,4-diamine as an oil.

### Part E

Under an atmosphere of argon, diethoxymethyl acetate (2.24 L, 13.7 mol) was added to a solution of N⁴-(2-methylpropyl)[1,5]naphthyridin-3,4-diamine (2.826 kg, 13.07 mol) in toluene (32.5 L) with continuous stirring while maintaining the reaction temperature at or below 30.3° C. The reaction mixture was stirred for 40 minutes at a temperature of 30.1° C. to 30.3° C., heated to reflux over a period of 45 minutes, heated at reflux (92.5° C. to 98.5° C.) for 185 minutes, and allowed to cool to room temperature overnight. Saturated aqueous potassium carbonate (6 L) was added, and the resulting mixture was stirred for 32 minutes at a temperature of 29.3° C. to 30.3° C. and subsequently allowed to stand for 53 minutes. The organic fraction was separated and concentrated under reduced pressure at a temperature of 55° C. to 65° C. over a period of seven hours. The resulting oil was triturated with heptane (3 L) at 20° C. to form a solid, which was isolated by filtration with agitation, washed with cold heptane (1.5 L at 5° C.) and allowed to air-dry to provide 2.593 kg of 1-(2-methylpropyl)-1H-imidazo[4,5-c][1,5]naphthyridine.

### Part F

A solution of 1-(2-methylpropyl)-1H-imidazo[4,5-c][1,5]naphthyridine (2.593 kg, 11.46 mol) in chloroform (2.8 L) was stirred for one hour at a temperature of 17.5° C. to 18° C., and then 3-chloroperoxybenzoic acid (2.864 g of 70% pure material, 11.6 mol) was added in five portions approximately five minutes apart. During the addition, the temperature of the reaction increased from 16.4° C. to 26.1° C. After the addition, the reaction mixture was stirred for 20 hours, and the reaction temperature decreased from 26.1° C. to 17.5° C. The reaction mixture was then stirred for thirty minutes with 1% w/w aqueous sodium carbonate (4×3.2 L, 30 minutes between washings). The organic fraction was concentrated under reduced pressure at 40° C. The residue (4.6 kg) was triturated with diethyl ether (7.5 L) for 68 minutes, and the resulting solid was isolated by filtration with agitation, washed with diethyl ether (4.5 L), and dried under suction to provide 3.304 kg of 1-(2-methylpropyl)-5-oxido-1H-imidazo[4,5-c][1,5]naphthyridine.

### Part G

Aqueous ammonium hydroxide (21 L of 28% by weight (w/w)) was added with continuous stirring to a solution of the material from Part F in dichloromethane (34 L) while maintaining the reaction temperature at or below 11.5° C. With continuous stirring, p-toluenesulfonyl chloride (1.786 kg, 9.368 mol) was added in portions over a period of one hour while maintaining the reaction temperature at 16.4° C. to 25° C. The reaction was stirred for 140 minutes, additional p-toluenesulfonyl chloride (180 g, 0.94 mol) was added, and the reaction was stirred for one additional hour. Water (21 L) was added to the reaction mixture, and the resulting mixture was stirred for 30 minutes and allowed to stand for 14.5 hours. The organic fraction was separated and concentrated under reduced pressure at 40° C. over a period of 8.25 hours. The residue (1.004 kg) was heated at reflux in acetonitrile (10.04 L) for 128 minutes. The suspension was allowed to cool to 20° C., and the resulting solid was isolated by filtration, washed with cold acetonitrile (1.4 L at 4° C.), and air-dried to provide 360 g of 1-(2-methylpropyl)-1H-imidazo[4,5-c][1,5]naphthyridin-4-amine. A solid was present in the reserved aqueous fraction, and the solid was isolated by filtration, washed with water (4×2000 mL), and dried under suction to provide 1.925 kg of 1-(2-methylpropyl)-1H-imidazo[4,5-c][1,5]naphthyridin-4-amine. The two solids were combined and heated at reflux in 90% w/w methanol/water (22.85 L) for 310 minutes, and the suspension was allowed to cool to 24.1° C. overnight. The resulting solid was isolated by filtration, washed with cold 90% w/w methanol/water (1.5 L at 5° C.), and dried in a vacuum oven at 75° C. and 1×10⁵ Pa for five days to provide 1.368 kg of 1-(2-methylpropyl)-1H-imidazo[4,5-c][1,5]naphthyridin-4-amine as a light yellow solid.

### Part H

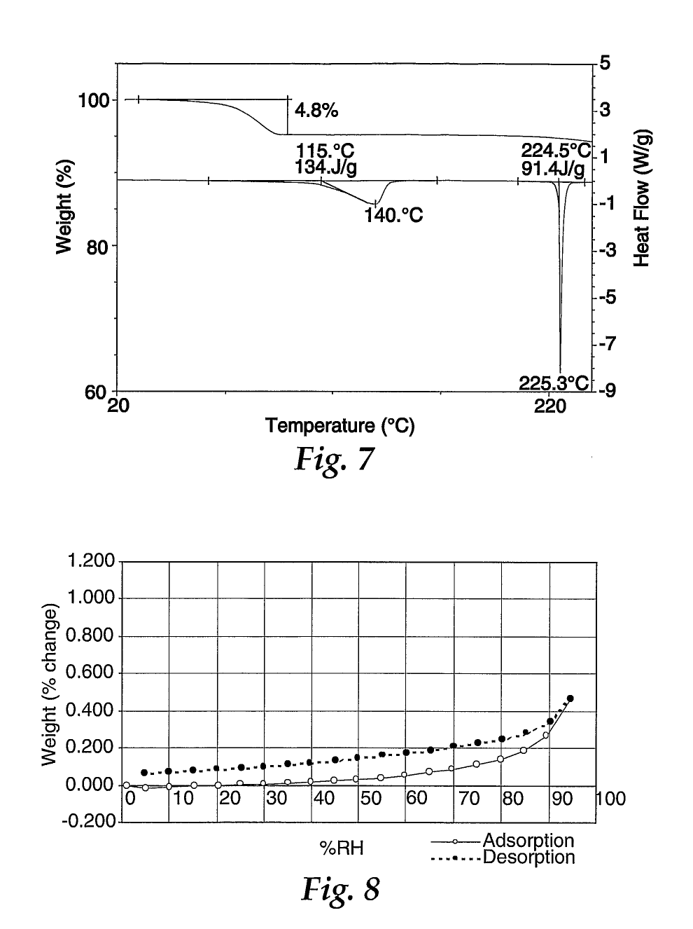

With stirring, 1-(2-methylpropyl)-1H-imidazo[4,5-c][1,5]naphthyridin-4-amine (300 g, 1.24 mol) followed by a rinse of 2% (v/v) water/isopropyl alcohol (100 mL) was added to a vessel containing 2% (v/v) water/isopropyl alcohol (2000 mL), and the resulting mixture was heated to 81° C. A solution of ethanesulfonic acid (151 g of 95%, 1.37 mol) in 2% (v/v) water/isopropyl alcohol (600 mL) was added slowly to the reaction mixture over a period of approximately 20 minutes. During the addition, the mixture became a clear solution, and the temperature was 81° C. to 82° C. The addition funnel was rinsed with 2% v/v water/isopropyl alcohol (320 mL); the temperature dropped during the addition but returned to reflux. The resulting solution was heated at reflux for 8 minutes and then cooled slowly to room temperature at a rate of 0.2° C./minute (57° C. over 234 minutes). The resulting slurry was then further cooled at a rate of 0.2° C./minute to a temperature of 0° C. to 5° C. (i.e., the slurry was cooled 21° C. over a period of 130 minutes). The solid was isolated by filtration using cold 2% (v/v) water/isopropyl alcohol (400 mL, 3.5° C.) to rinse and aid in the transfer. The solid was washed with cold 2% (v/v) water/isopropyl alcohol (300 mL, 3.5° C.), dried at about 43° C. under 1.69×10⁴ Pa to 1.70×10⁴ Pa (169 mbar to 170 mbar) for 23 hours and 40 minutes to provide 455 g of 1-(2-methylpropyl)-1H-imidazo[4,5-c][1,5]naphthyridin-4-amine ethanesulfonate monohydrate. The material was processed in a Hamilton Beach 14-speed blender to provide a white “cotton-like” solid, mp 221.5° C.-223.4° C. Anal. Calcd. for C₁₃H₁₅N₅.C₂H₆O₃S.H₂O: C, 48.77; H, 6.28; N, 18.96. Found: C, 48.70; H, 6.25; N, 18.96. This material was characterized by powder X-ray diffraction analysis, ¹³C NMR spectroscopy, water sorption analysis, thermogravimetric analysis, and differential scanning calorimetry. The powder X-ray diffraction pattern and ¹³C NMR spectra are shown in FIGS. 2 and 3. The DSC/TGA overlay is shown in FIG. 7, and the water sorption isotherm is shown in FIG. 6.

### Example 2

### Preparation of 1-(2-Methylpropyl)-1H-imidazo[4,5-c][1,5]naphthyridin-4-amine ethanesulfonate monohydrate

### Part A

Under an argon atmosphere, a solution of 3-nitro[1,5]naphthyridin-4-ol (0.800 kg, 4.19 mol) in DMF (8 L) was cooled to 19° C. Phosphorous oxychloride (803.6 g, 5.24 mol) was added over a period of 70 minutes while maintaining the temperature at 19° C. to 20° C. A precipitate formed, and the reaction was stirred overnight at room temperature. The reaction mixture was divided in two, and each half was then added with stirring to ice water (13 to 15 L) while maintaining the temperature below 15° C. The resulting mixtures were stirred for 30 minutes, and the resulting solids were isolated by filtration, washed with cold water (2 L), and combined. The solid was then mixed with dichloromethane (about 9 L) and heated to 30° C. The organic fraction was removed, and the rest of the mixture was filtered through a layer of CELITE filter agent. The filter cake was washed with dichloromethane (2 L). The filtrate was combined with the organic fraction, and the resulting solution was allowed to stand over sodium sulfate until it was used in Part B.

### Part B

The mixture from Part A was filtered. Under a nitrogen atmosphere and with stirring, isobutylamine (766.2 g, 10.48 millimoles (mmol)) was added to the filtrate over a period of 95 minutes while maintaining a reaction temperature of 19° C. to 25° C. The reaction was stirred overnight at room temperature and then concentrated to dryness under reduced pressure. The resulting solid was stirred with deionized water (16 L) overnight at room temperature, isolated by filtration, and dried under vacuum for 26.5 hours at 62° C. to provide 0.92 kg of N⁴-(2-methylpropyl)-3-nitro[1,5]naphthyridin-4-amine as a yellow solid. The product was split into three portions.

### Part C

A Parr vessel was charged with toluene (2.3 L), 2-propanol (230 mL), N⁴-(2-methylpropyl)-3-nitro[1,5]naphthyridin-4-amine (230 g, 0.924 mol), and 5% platinum on carbon (23.0 g). The vessel was sealed and purged three times with nitrogen while the reaction mixture was shaken. The reaction mixture was then placed under hydrogen pressure (3.4×10⁵ Pa, 50 psi) overnight. Additional 5% platinum on carbon (7 g) was added, and the reaction was placed under hydrogen pressure (3.4×10⁵ Pa, 50 psi) overnight. The reaction mixture was filtered through a layer of CELITE filter agent. The filter cake was washed with 10:1 toluene/2-propanol, and the filtrate was concentrated under reduced pressure to provide 201.7 g of N⁴-(2-methylpropyl)[1,5]naphthyridine-3,4-diamine as a dark oil. The material was mixed with material from two other runs and used in Part D.

### Part D

Under an atmosphere of nitrogen, pyridine hydrochloride (6.94 g, 56.5 mmol) was added to a solution of N⁴-(2-methylpropyl)[1,5]naphthyridin-3,4-diamine (642.0 g, 2.97 mol) in toluene (3.3 L), and the resulting mixture was stirred until a homogenous solution was achieved. The solution was heated to 90° C., and triethyl orthoformate (484.6 g, 3.27 mol) was added. When the reaction temperature had reached 100° C., the addition was stopped, and additional pyridine hydrochloride (6.94 g, 56.5 mmol) was added. The addition was resumed and carried out over a period of 65 minutes while maintaining a reaction temperature in the range of 93° C. to 105° C. and removing the volatiles formed during the reaction by distillation. The reaction was heated at about 90° C. for 25 minutes, allowed to cool to room temperature, and allowed to stir overnight. Additional pyridine hydrochloride (2 g) was then added and the reaction was heated to 112° C. until 2.4 L of distillate were collected. The reaction was then allowed to cool to room temperature, diluted with toluene, stirred for two days, and washed with aqueous sodium carbonate (3 L of 5%). The organic fraction was separated and dried over magnesium sulfate, filtered, and concentrated under reduced pressure. The resulting dark oil was triturated with hexane (1.5 L) to form a solid, which was collected by filtration, washed with hexane (250 mL), and dried under vacuum at a temperature of 50° C. to 52° C. to provide 440.4 g of 1-(2-methylpropyl)-1H-imidazo[4,5-c][1,5]naphthyridine as a tan solid. A portion of this material was mixed with material from another run and used in Part E.

### Part E

A solution of 1-(2-methylpropyl)-1H-imidazo[4,5-c][1,5]naphthyridine (554.3 g, 2.45 mol) in dichloromethane (5.54 L) was cooled to 6° C., and 3-chloroperoxybenzoic acid (665 g of 70% pure material, 2.70 mmol) was added in portions (75 g each) every ten minutes while maintaining the reaction temperature at or below 20° C. to provide a solution of 1-(2-methylpropyl)-5-oxido-1H-imidazo[4,5-c][1,5]naphthyridine in dichloromethane.

### Part F

Aqueous ammonium hydroxide (5 L of 28% w/w) was added with continuous stirring to the solution from Part E, and the mixture was cooled to a temperature of 8° C. to 10° C. With continuous stirring, a solution of p-toluenesulfonyl chloride (506 g, 2.70 mol) in dichloromethane (2 L), which had been filtered through a 10- to 20-micron porous fritted filter, was added slowly over a period of 55 minutes while maintaining the reaction temperature at 14° C. to 16° C. The reaction was stirred for about 48 hours; it warmed to 20° C. during this time. The reaction mixture was cooled to a temperature of 3.5° C., and the resulting solid was isolated by filtration and washed sequentially with cold dichloromethane (500 mL) and water (2 L). The solid was stirred in an aqueous solution (3 L) of sodium carbonate (125 g) and sodium thiosulfate (35 g) for five hours, isolated by filtration, and washed with deionized water. The solids were stirred overnight in deionized water (2 L), isolated by filtration, washed with deionized water (200 mL), and dried under vacuum at 58° C. to 64° C. for 24 hours to provide 384.8 g of 1-(2-methylpropyl)-1H-imidazo[4,5-c][1,5]naphthyridin-4-amine, which was mixed with material from another run to provide 721 g of product. The product was heated to reflux in 90% w/w methanol/water (3.605 L) over 30 minutes, heated at reflux for 150 minutes, and allowed to cool slowly to 20° C. overnight. The resulting solid was isolated by filtration, washed with 90% w/w methanol/water (300 mL), and dried in a vacuum oven at 70° C. for 24 hours to provide 705.6 g of 1-(2-methylpropyl)-1H-imidazo[4,5-c][1,5]naphthyridin-4-amine as an off-white solid.

### Part G

A mixture of 1-(2-methylpropyl)-1H-imidazo[4,5-c][1,5]naphthyridin-4-amine (100.0 g, 0.414 mol) and 2% (v/v) deionized water/isopropyl alcohol (700 mL) was heated to reflux with moderate stirring, and ethanesulfonic acid (95% purity, 52.8 g, 0.456 mol) was added over a period of ten minutes using an addition funnel. The funnel was rinsed with 2% (v/v) deionized water/isopropyl alcohol (300 mL), and the resulting solution was heated at reflux for 30 minutes. The solution was stirred and allowed to cool overnight at a rate of 0.5° C./minute to a temperature of 28° C. The resulting thick suspension was further cooled to 21° C. for 50 minutes, and the solid was collected by vacuum filtration, washed with 2% (v/v) deionized water/isopropyl alcohol (300 mL), and dried under vacuum for 21.5 hours at 46° C. to 47° C. to provide 142.0 g of 1-(2-methylpropyl)-1H-imidazo[4,5-c][1,5]naphthyridin-4-amine ethanesulfonate monohydrate as a white solid, mp 220.0-221.0° C. Anal. Calcd. for C₁₃H₁₅N₅.C₂H₆O₃S.H₂O: C, 48.77; H, 6.28; N, 18.96. Found: C, 48.76; H, 6.53; N, 19.01. This material was characterized by powder X-ray diffraction analysis and the data were found to be consistent with FIG. 2.

### Example 3

### Preparation of 1-(2-Methylpropyl)-1H-imidazo[4,5-c][1,5]naphthyridin-4-amine ethanesulfonate monohydrate

1-(2-Methylpropyl)-1H-imidazo[4,5-c][1,5]naphthyridin-4-amine prepared in Example 1, Parts A through G was used as the starting material for this example. A mixture of 1-(2-methylpropyl)-1H-imidazo[4,5-c][1,5]naphthyridin-4-amine (2.0 g, 8.3 mmol) and 2% (v/v) deionized water/isopropyl alcohol (20 mL) was heated to reflux with moderate stirring, and ethanesulfonic acid (1.0 g, 9.1 mmol) was added followed by a 2% (v/v) deionized water/isopropyl alcohol (6 mL) rinse, and the resulting solution was heated at reflux for 15 minutes. The solution was stirred and allowed to cool at a rate of 0.5° C./minute for one hour, 0.33° C./minute during the second hour, and 0.1° C./minute during the third hour. Stirring was continued for 22 hours, and the temperature reached 20° C. The resulting solid was collected by vacuum filtration, washed with 2% (v/v) deionized water/isopropyl alcohol (15 mL), and dried under vacuum (1.6×10³ Pa to 2.1×10³ Pa) (12 mmHg to 16 mmHg) for 20 hours at 45° C. to 46° C. to provide 2.79 g of 1-(2-methylpropyl)-1H-imidazo[4,5-c][1,5]naphthyridin-4-amine ethanesulfonate monohydrate as a white solid. Anal. Calcd. for C₁₃H₁₅N₅.C₂H₆O₃S.H₂O: C, 48.76; H, 6.30; N, 18.96. Found: C, 48.76; H, 6.43; N, 18.99. This material was characterized by powder X-ray diffraction analysis, DSC, TGA, and water sorption analysis, and the data were found to be consistent with those shown in FIGS. 2, 7, and 6, respectively.

### Example 4

### Preparation of 1-(2-Methylpropyl)-1H-imidazo[4,5-c][1,5]naphthyridin-4-amine ethanesulfonate monohydrate

1-(2-Methylpropyl)-1H-imidazo[4,5-c][1,5]naphthyridin-4-amine prepared in Example 1, Parts A through G was used as the starting material for this example. A mixture of 1-(2-methylpropyl)-1H-imidazo[4,5-c][1,5]naphthyridin-4-amine (2.0 g, 8.3 mmol) and 2% (v/v) deionized water/isopropyl alcohol (20 mL) was heated to reflux with moderate stirring, and ethanesulfonic acid (1.0 g, 9.1 mmol) was added followed by a 2% (v/v) deionized water/isopropyl alcohol (6 mL) rinse, and the resulting solution was heated at reflux for 15 minutes. The solution was stirred and allowed to cool at a rate of 0.25° C./minute for two hours, 0.23° C./minute during the third hour, and 0.13° C./minute during the fourth hour. Stirring was continued for 45 hours, and the temperature reached 24° C. The resulting solid was collected by vacuum filtration, washed with 2% (v/v) deionized water/isopropyl alcohol (15 mL), and dried under vacuum (2.6×10² Pa to 2.1×10³ Pa) (2 mmHg to 16 mmHg) for 20 hours at 46° C. to 47° C. to provide 2.74 g of 1-(2-methylpropyl)-1H-imidazo[4,5-c][1,5]naphthyridin-4-amine ethanesulfonate monohydrate as a white solid. This material was characterized by powder X-ray diffraction analysis, DSC, TGA, and water sorption analysis, and the data were found to be consistent with those shown in FIGS. 2, 7, and 6, respectively.

### Example 5

### Preparation of 1-(2-Methylpropyl)-1H-imidazo[4,5-c][1,5]naphthyridin-4-amine ethanesulfonate monohydrate

1-(2-Methylpropyl)-1H-imidazo[4,5-c][1,5]naphthyridin-4-amine prepared in Example 1, Parts A through G was used as the starting material for this example. A mixture of 1-(2-methylpropyl)-1H-imidazo[4,5-c][1,5]naphthyridin-4-amine (2.0 g, 8.3 mmol) and 2% (v/v) deionized water/ethyl acetate (14 mL) was heated to reflux with moderate stirring, and ethanesulfonic acid (1.0 g, 9.1 mmol) was added followed by a 2% (v/v) deionized water/ethyl acetate (6 mL) rinse, and the resulting suspension was heated at reflux for 15 minutes and allowed to cool overnight. The resulting solid was collected by vacuum filtration, washed with 2% (v/v) deionized water/ethyl acetate (15 mL), and dried under vacuum (2.6×10² Pa to 2.1×10³ Pa) (2 mmHg to 16 mmHg) for 21 hours at 46° C. to 47° C. to provide 2.91 g of 1-(2-methylpropyl)-1H-imidazo[4,5-c][1,5]naphthyridin-4-amine ethanesulfonate monohydrate as white needles. Anal. Calcd. for C₁₃H₁₅N₅.C₂H₆O₃S.H₂O: C, 48.76; H, 6.30; N, 18.96. Found: C, 48.48; H, 6.47; N, 18.93. This material was characterized by powder X-ray diffraction analysis, DSC, TGA, and water sorption analysis, and the data were found to be consistent with those shown in FIGS. 2, 7, and 6, respectively.

### Example 6

### Preparation of 1-(2-Methylpropyl)-1H-imidazo[4,5-c][1,5]naphthyridin-4-amine ethanesulfonate monohydrate

### Part A

Under a nitrogen atmosphere, a suspension of 3-nitro-1,5-naphthyridin-4-ol (12.00 kg, 67.78 mol) in DMF (49 L) was stirred for 30 minutes at a temperature of 20° C. to 24° C. Phosphorous oxychloride (10.6 kg, 69.1 mol) was added slowly over a period of 53 minutes while maintaining the temperature at 20.6° C. to 25.6° C. Additional DMF (5 L) was used to rinse the addition vessel and added to the reaction. The reaction was stirred for 19 hours and 17 minutes at a temperature of 20° C. to 24° C. and then added quickly, over a period of four minutes, to purified water (275 L) that had been cooled to 8.4° C. During the addition, the temperature of the mixture did not exceed 18° C. Additional water (80 L) was used to rinse the original vessel and added quickly to the resulting mixture, which ranged in temperature from 16.6° C. to 17.2° C. during this addition. The mixture resulting from the additions was stirred for 30 minutes while cooling to a temperature of approximately 10° C. A solid formed and was isolated by filtration and washed with cold water (6×33 L at 10° C.) to provide 20.55 kg of 4-chloro-3-nitro[1,5]naphthyridine, which contained some water and was used in Part B within 2.75 hours of filtration.

### Part B

Isobutylamine (9.4 kg, 12.8 L, 130 mol) was added to a stirred suspension of the material from Part A (20.55 kg) in tetrahydrofuran (67 L) over a period of 77 minutes while maintaining a reaction temperature of 20° C. to 27° C. The addition of isobutylamine was followed by a rinse with tetrahydrofuran (5 L). The reaction was stirred for 190 minutes at a temperature of 20° C. to 24° C., and then water (288 L) was added over a period of about one hour while maintaining the reaction temperature at 21.4° C. to 23.8° C. The resulting mixture was stirred at 20° C. to 24° C. for 75 minutes and then filtered. The isolated solid was washed with water (4×25 L) that had also been used to rinse the reaction vessel, pulled dry under vacuum, and further dried under vacuum for 60 hours at a temperature of 45° C. to 55° C. to provide 13.7 kg of N⁴-(2-methylpropyl)-3-nitro[1,5]naphthyridin-4-amine.

### Part C

A hydrogenation vessel was charged with N⁴-(2-methylpropyl)-3-nitro[1,5]naphthyridin-4-amine (13.7 kg, 55.6 mol) and 3% platinum on carbon (0.79 kg, 34.61% w/w in water), purged with nitrogen, charged with toluene (670.0 kg) and 2-propanol (37.0 kg), and purged with nitrogen. The vessel was sealed and purged three times with hydrogen. The reaction mixture was then placed under hydrogen pressure (1.2×10⁵ Pa, 17 psi) for 170 minutes while stirring and maintaining the temperature between 18° C. and 22° C. The reaction mixture was filtered through a filter pad. The filter cake was washed with toluene (87 kg), and the filtrate was concentrated under reduced pressure at approximately 25° C. to provide a solution of N⁴-(2-methylpropyl)[1,5]naphthyridine-3,4-diamine in toluene (approximately 15 mL/g).

### Part D

The solution from Part C (196 L) was cooled to a temperature of −5° C. to 0° C. under an atmosphere of nitrogen, and p-toluenesulfonic acid (0.520 kg, 2.73 mol) was added. The reaction was purged with nitrogen and heated to a temperature of 88° C. to 92° C. over a period of 87 minutes. Triethyl orthoformate (9.4 kg, 63 mol) was slowly added with stirring over a period of 64 minutes; during the addition, the reaction temperature was increased from 89.4° C. to 94° C. The addition vessel was rinsed with toluene (5 L), which was added to the reaction. The reaction mixture was stirred and heated at reflux (98° C. to 100° C.) for 195 minutes; the ethanol distillate was collected. The reaction was purged three times with nitrogen, cooled to a temperature of 0° C. to 5° C. over a period of three hours, purged three times with nitrogen, and heated to a temperature of 20° C. to 24° C. The reaction mixture was washed sequentially with aqueous sodium carbonate (41 L of 1% w/w) for one hour and water (41 L) for one hour, while allowing the layers to separate for one hour after each washing. The organic fraction was then concentrated under reduced pressure at a temperature of 21.6° C., cooled to a temperature of 0° C. to 5° C., and purged with nitrogen to provide 1-(2-methylpropyl)-1H-imidazo[4,5-c][1,5]naphthyridine as a solution in toluene (approximately 7.3 mL/g).

### Part E

The solution from Part D (102 L) was heated to a temperature of 45° C. to 50° C. and purged with nitrogen. Under a nitrogen atmosphere, peracetic acid (12.1 kg of 40 weight percent (wt. % or w/w)) was added over a period of 137 minutes while maintaining the reaction temperature at 45.6° C. to 50° C. and purging the system with nitrogen every 30 minutes. The addition vessel was rinsed with toluene (5 L), which was added to the reaction. The reaction was stirred for 247 minutes at a temperature of 47.5° C. to 49.7° C., while purging the system with nitrogen every thirty minutes, and then cooled to a temperature of 0° C. to 5° C. The reaction was purged with nitrogen and heated to a temperature of 43° C. to 47° C. While maintaining this temperature, aqueous sodium metabisulfite (51 L of 4% w/w) was added over a period of 28 minutes, and aqueous sodium hydroxide (50 L of 10.5% w/w) was added over a period of 28 minutes. Water (7 L) was added, and the reaction temperature was adjusted to 48° C. to 52° C. and stirred for 30 minutes. The resulting mixture was cooled to a temperature of 3° C. to 7° C. over a period of about four hours, and the reaction was maintained at this temperature for 249 minutes. A solid was present and was isolated by filtration, washed with cold water (3×25 L at 3° C. to 7° C.), dried by suction, and further dried under vacuum at 25° C. to 35° C. for about 137 hours to provide 10.8 kg of 1-(2-methylpropyl)-5-oxido-1H-imidazo[4,5-c][1,5]naphthyridine as a yellow solid.

### Part F

Under a nitrogen atmosphere, aqueous ammonium hydroxide (11.5 kg of 28% w/w) was quickly added with continuous stirring to a suspension of 1-(2-methylpropyl)-5-oxido-1H-imidazo[4,5-c][1,5]naphthyridine (10.8 kg, 44.6 mol) in methanol (77 L) while maintaining the reaction temperature at 20.6° C. to 21.4° C. Methanol (10 L) was used to rinse the addition vessel and added to the reaction. The reaction was stirred for 29 minutes. With continuous stirring, benzenesulfonyl chloride (8.2 kg, 46 mol) was added over a period of 50 minutes while maintaining the reaction temperature at 20° C. to 29.6° C. Methanol (10 L) was used to rinse the addition vessel and added to the reaction. The reaction was stirred for 66 minutes at 20.4° C. to 22.5° C. and purged three times with nitrogen. Additional benzenesulfonyl chloride (0.8 kg, 4.5 mol) was added over a period of 12 minutes while maintaining the reaction temperature at 22.9° C. to 23.7° C., and additional methanol (5 L) was added. The reaction was then stirred for 67 minutes at a temperature of 21.6° C. to 24.1° C. Aqueous sodium hydroxide (32 L of 10% w/w) was added to the reaction mixture over a period of 30 minutes while maintaining the reaction temperature between 22° C. and 23° C. Water (10 L), used to rinse the addition vessel, was added to the reaction mixture. The resulting mixture was cooled to 12° C. over a period of 39 minutes and stirred for two hours at a temperature in the range of 10.6° C. and 12.0° C. A precipitate was present and was isolated by filtration and washed with cold (8° C. to 12° C.) 59:41 methanol/water (2×11 L), allowing the wash to soak into the filter cake for 10 minutes with each wash. The filter cake was washed with cold (8° C. to 12° C.) water (4×25 L), allowing the wash to soak into the filter cake for 10 minutes with each wash, and dried under vacuum at a temperature of 45° C. to 55° C. for 24 hours to provide 8.25 kg of 1-(2-methylpropyl)-1H-imidazo[4,5-c][1,5]naphthyridin-4-amine as a light yellow solid. A portion of the solid (2.75 kg) was mixed with 2-butanol (371 L), and the resulting mixture was heated to reflux with stirring over a period of 93 minutes, heated at 99° C. for 20 minutes, cooled to a temperature of 60° C. to 65° C. over a period of 45 minutes, and filtered into another warm vessel. The solution was then cooled to a temperature of 20° C. to 25° C. with stirring, concentrated under reduced pressure at a temperature of 21.4° C. to a minimum volume required for stirring, and purged with nitrogen. A second portion of 1-(2-methylpropyl)-1H-imidazo[4,5-c][1,5]naphthyridin-4-amine as a light yellow solid (2.75 kg) and a second volume of 2-butanol (371 L) were added, and the refluxing, filtering, and concentration processes were repeated. A third portion of 1-(2-methylpropyl)-1H-imidazo[4,5-c][1,5]naphthyridin-4-amine as a light yellow solid (2.60 kg) and a third volume of 2-butanol (371 L) were added, and the refluxing and filtering processes were repeated. The solution was cooled to 20° C., and additional 2-butanol (20 L) was added. The refluxing, filtering, and concentration processes were repeated. The resulting mixture (198 L total) was heated to reflux with stirring over a period of 70 minutes, heated at 97° C. for 20 minutes, cooled at a rate of 0.5° C. per minute to a temperature of 68° C. to 72° C., cooled to a temperature of 3° C. to 5° C. over a period of 154 minutes, stirred at a temperature of 4.3° C. to 5° C. for two hours, and filtered with no agitation. The filter cake was washed with cold (3° C. to 5° C.) 2-butanol (50 L) that had been used to wash the crystallization vessel and blown dry without agitation for one hour under a nitrogen flow. The solid was dried on the filter with no agitation at a temperature of 46° C. to 50° C. under vacuum (900 mbar to 980 mbar, 9.0×10⁴ Pa to 9.8×10⁴ Pa) with small nitrogen sweep for twelve hours to provide 6.50 kg of 1-(2-methylpropyl)-1H-imidazo[4,5-c][1,5]naphthyridin-4-amine.

### Part G

Under a nitrogen atmosphere, isopropyl alcohol (88 L) was added to 1-(2-methylpropyl)-1H-imidazo[4,5-c][1,5]naphthyridin-4-amine (3.45 kg, 14.3 mol), and the mixture was stirred and heated to 81° C. Aqueous ethanesulfonic acid (2.5 kg of 70% w/w, 16 mol) was added over a period of 15 minutes, and the addition vessel was rinsed with isopropyl alcohol (10 L). The mixture was heated at reflux for 30 minutes, cooled to a temperature of 60° C. to 65° C., and filtered through a 5-micron filter into another warm vessel. Heated isopropyl alcohol (temperature of 60° C. to 65° C.) was used to rinse the first vessel and added to the solution. The filtrate was heated at reflux (81° C.) for 37 minutes (min) and then cooled to a temperature of 45° C. to 55° C. over the course of 85 minutes (approximately 0.4° C./min). The reaction was further cooled to a temperature of 23° C. to 27° C. over 130 minutes (approximately 0.15° C./min to 0.25° C./min) and then stirred at a temperature of 23° C. to 27° C. for 100 minutes before the addition of tert-butyl methyl ether (MTBE) (123 mL) over a period of 92 minutes. The resulting mixture was stirred at a temperature of 24° C. to 25° C. for 63 minutes, cooled to 4° C. over 115 minutes (approximately 0.18° C./min), and stirred at a temperature of 4° C. to 5° C. for 125 minutes. The slurry was transferred over a period of 11 minutes to a nitrogen-purged filter in 2 portions, using nitrogen pressure to assist filtration. MTBE (88 L) was added to the crystallization vessel, cooled to a temperature of 1° C. to 5° C. and then transferred to the filter to wash the product cake. No agitation was used during the filtration. The solid was dried on the filter with no agitation at a temperature of 35° C. to 45° C. under vacuum (900 mbar to 980 mbar, 9.0×10⁴ Pa to 9.8×10⁴ Pa) with small nitrogen sweep for six hours to provide 3.8 kg of a solid. The solid was slurried in a water (400 mL)/isopropyl alcohol (1 L)/MTBE (15 L) mixture for 24 to 36 hours. The slurry was filtered and washed with MTBE. The product was dried at 40° C. under vacuum (980 mbar, 9.8×10⁴ Pa) to provide 1-(2-methylpropyl)-1H-imidazo[4,5-c][1,5]naphthyridin-4-amine ethanesulfonate monohydrate as a white solid. This material was characterized by powder X-ray diffraction analysis and FTIR spectroscopy. The powder X-ray diffraction pattern was found to be consistent with FIG. 2, and the FTIR spectrum is shown in FIG. 4.

### Example 7

### Preparation of 1-(2-Methylpropyl)-1H-imidazo[4,5-c][1,5]naphthyridin-4-amine methanesulfonate monohydrate

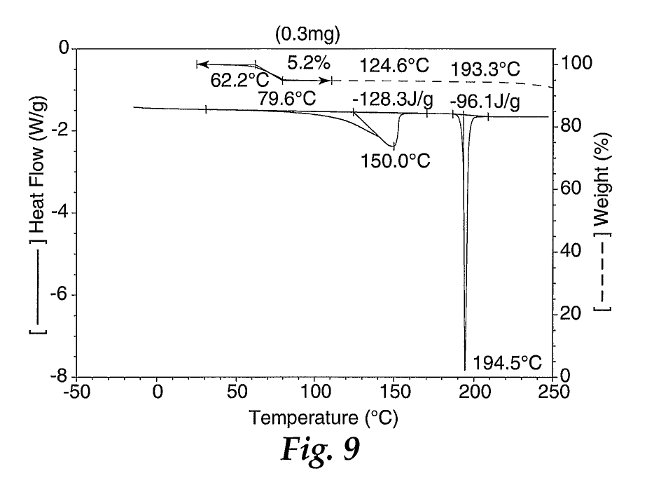

A mixture of 1-(2-methylpropyl)-1H-imidazo[4,5-c][1,5]naphthyridin-4-amine (1.0 g, 4.1 mmol) and 2% (v/v) deionized water/isopropyl alcohol (7 mL) was heated to reflux with stirring, and a solution of methanesulfonic acid (0.44 g, 4.6 mmol) in 2% (v/v) deionized water/isopropyl alcohol (3 mL). The solution was heated at reflux briefly and then allowed to cool to room temperature. The resulting mixture was further cooled to a temperature of 0° C. to 5° C., and the solid was collected by vacuum filtration and washed with cold isopropyl alcohol (5 mL). The isolated solid was a dense mat of needles, which was cut into pieces 30 millimeters (mm) to 60 mm wide and dried under vacuum (1.3×10³ Pa, 10 mmHg) for 24 hours at 45° C. to provide 1.33 g of 1-(2-methylpropyl)-1H-imidazo[4,5-c][1,5]naphthyridin-4-amine methanesulfonate monohydrate as white needles. Anal. Calcd. for C₁₃H₁₅N₅.C₂H₆O₃S.0.6H₂O: C, 47.31; H, 5.96; N, 19.71. Found: C, 48.26; H, 5.76; N, 20.32. This material was characterized by powder X-ray diffraction analysis, DSC, TGA, and water sorption analysis. The powder X-ray diffraction pattern is shown in FIG. 1. The DSC/TGA overlay is shown in FIG. 9, and the water sorption isotherm is shown in FIG. 8.

### In-Situ Salt Selection Test

Aqueous solutions (0.5 molar (M)) of ethanesulfonic acid, methanesulfonic acid, and hydrochloric acid were prepared. For ethanesulfonic acid and methanesulfonic acid, the neat acid (0.05 mol) was dissolved in water (100 mL) to prepare the solutions. Hydrochloric acid (1 normal (N)) was diluted with water to prepare 0.5 M hydrochloric acid. The free base of 1-(2-methylpropyl)-1H-imidazo[4,5-c][1,5]naphthyridin-4-amine in an amount indicated in the following Table 1 was added to a vial, and the acidic solution from the following Table 1 (1.05 equivalent) was added. The vial was capped and shaken at 25° C. in a water bath for 24 hours. The appearance of the resulting solution was evaluated, and the volume of water indicated in the following Table 1 was added. The resulting salt mixture or solution was filtered through Whatman No. 5 filter paper, and 1 mL of the filtrate was diluted with methanol (10 mL) to provide a specimen for analysis. The solubility of the salt in water was determined for each specimen by analyzing for soluble salt content by reverse phase high performance liquid chromatography (HPLC) using the following method.

A diluent was prepared by dissolving phosphoric acid (1 mL of 85%) in HPLC grade water (1 L). The resulting solution was mixed in a 60:40 ratio with acetonitrile. Each specimen was diluted to a volume of 50.0 mL with the diluent to provide a sample having a concentration of less than or equal to (≦) 50 micrograms per milliliter (μg/mL).

An external standard was prepared by dissolving 1-(2-methylpropyl)-1H-imidazo[4,5-c][1,5]naphthyridin-4-amine free base (25 mg) in methanol (30 mL) with the aid of sonication and then diluting to a volume of 50.0 mL with methanol to provide a stock solution. Diluent was added to 5 mL of the stock solution to a volume of 50.0 mL.

A Waters LC Module 1 HPLC system equipped with an autosampler, a reproducible injector, a UV detector (326 nm wavelength), and Turbochrom data acquisition software was used with a ZORBAX Bonus-RP C₁₄, 15 cm×4.6 mm column with 5 μm packing. The mobile phase was prepared by dissolving 1-octanesulfonate, sodium salt (2.00 g) and phosphoric acid (1 mL of 85%) in HPLC grade water (1 L) and mixing the resulting solution in a 65:35 ratio with HPLC grade acetonitrile. A flow rate of 1.0 mL/min was used.

The column was equilibrated with the mobile phase, and the system was tested using six 20 μL injections of the external standard to ensure that the relative standard deviation for the peak areas was less than or equal to (≦) 1.5%. An injection of each sample (20 μL) was tested in comparison to a standard. The concentration, in percentage, of 1-(2-methylpropyl)-1H-imidazo[4,5-c][1,5]naphthyridin-4-amine in solution was then calculated using the following operation: {[peak area of the sample/weight of the sample (mg)]/[peak area of the standard/weight of the standard (mg)]average}×100. The solubility of the salt, presented in the following Table 1, can then be calculated using the equations presented by Tong, W. Q., and Whitesell, G, Pharmaceutical Development and Technology, 3(2), pp. 215-223 (1998).

TABLE 1
In-situ salt selection test carried out in water

| | | | H₂O | |
|---|---|---|---|---|
| | | | Added | |
| | | | (mL), final | |
| Free Base | Acid (volume in | | concen- | Solubility |
| g/mmol | mL) | Appearance | tration (M) | (mg/mL) |
| 0.102/0.422 | Ethanesulfonic | Clear | 3.334, 0.1 | 24.4 |
| | (0.886) | | | |
| 0.101/0.419 | Methanesulfonic | Translucent | 4.000, 0.085 | 15.2 |
| | (0.881) | | | |
| 0.108/0.447 | Hydrochloric | Milky, | 8.002, 0.05 | 0.39 |
| | (0.938) | slightly | | |
| | | viscous | | |

The in-situ salt selection test was also used to determine solubilities of 1-(2-methylpropyl)-1H-imidazo[4,5-c][1,5]naphthyridin-4-amine ethanesulfonate and 1-(2-methylpropyl)-1H-imidazo[4,5-c][1,5]naphthyridin-4-amine methanesulfonate in mixtures of 5% (w/w) propylene glycol in water and 5% (w/w) diethylene glycol monoethyl ether in water. The method described above was used, with 5% (w/w) cosolvent in water added in lieu of water, and the data is provided in the following Table 2. For each sample, a final acid concentration of 0.05 M was obtained.

TABLE 2
In-situ salt selection test carried out in water with a cosolvent

| | | | H₂O | |
|---|---|---|---|---|
| | | | Added | Solu- |
| | | | (mL), | bility |
| Free Base | Acid (volume in | | cosolvent | (mg/ |
| g/mmol | mL) | Cosolvent | added (mL) | mL) |
| 0.114/0.472 | Ethanesulfonic | propylene | 8.919, 0.575 | greater |
| | (0.994) | glycol | | than |
| | | | | 10.1 |
| 0.116/0.481 | Methanesulfonic | propylene | 9.090, 0.547 | 10.3 |
| | (1.010) | glycol | | |
| 0.113/0.469 | Ethanesulfonic | diethylene | 8.865, 0.518 | 10.5 |
| | (0.984) | glycol | | |
| | | monoethylether | | |
| 0.120/0.495 | Methanesulfonic | diethylene | 9.36, 0.547 | 10.5 |
| | (1.04) | glycol | | |
| | | monoethylether | | |

### Cytokine Induction in Human Cells

1-(2-Methylpropyl)-1H-imidazo[4,5-c][1,5]naphthyridin-4-amine ethanesulfonate has been found to modulate cytokine biosynthesis by inducing the production of interferon α and/or tumor necrosis factor α when tested using the method described below.

An in vitro human blood cell system is used to assess cytokine induction. Activity is based on the measurement of interferon (α) and tumor necrosis factor (α) (IFN and TNF, respectively) secreted into culture media as described by Testerman et al. in “Cytokine Induction by the Immunomodulators Imiquimod and S-27609,” Journal of Leukocyte Biology, 58, 365-372 (September, 1995).

### Blood Cell Preparation for Culture

Whole blood from healthy human donors is collected by venipuncture into EDTA vacutainer tubes. Peripheral blood mononuclear cells (PBMC) are separated from whole blood by density gradient centrifugation using HISTOPAQUE-1077. Blood is diluted 1:1 with Dulbecco's Phosphate Buffered Saline (DPBS) or Hank's Balanced Salts Solution (HBSS). The PBMC layer is collected and washed twice with DPBS or HBSS and resuspended at 4×10⁶ cells/mL in RPMI complete. The PBMC suspension is added to 48 well flat bottom sterile tissue culture plates (Costar, Cambridge, Mass. or Becton Dickinson Labware, Lincoln Park, N.J.) containing an equal volume of RPMI complete media containing test compound.

### Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM.

### Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

### Separation

Following incubation the plates are centrifuged for 10 minutes at 1000 revolutions per minute (rpm) (approximately 200×g) at 4° C. The cell-free culture supernatant is removed with a sterile polypropylene pipet and transferred to sterile polypropylene tubes. Samples are maintained at −30° C. to −70° C. until analysis. The samples are analyzed for interferon (α) by ELISA and for tumor necrosis factor (α) by ELISA or IGEN Assay.

### Interferon (α) and Tumor Necrosis Factor (α) Analysis by ELISA

Interferon (α) concentration is determined by ELISA using a Human Multi-Species kit from PBL Biomedical Laboratories, New Brunswick, N.J. Results are expressed in pg/mL.

Tumor necrosis factor (α) (TNF) concentration is determined using ELISA kits available from Biosource International, Camarillo, Calif. Alternately, the TNF concentration can be determined by ORIGEN M-Series Immunoassay and read on an IGEN M-8 analyzer from IGEN International, Gaithersburg, Md. The immunoassay uses a human TNF capture and detection antibody pair from Biosource International, Camarillo, Calif. Results are expressed in pg/mL.

### Cytokine Induction Intravaginally

In the examples of the gel formulations below the serum and intravaginal cytokine data were obtained using the following general test method.

Rats were acclimated to collars (Lomir Biomedical, Malone, N.Y.) around the neck on two consecutive days prior to actual dosing. Rats were collared to prevent ingestion of the drug. Animals were then dosed intravaginally with 50 μL of gel. Rats received one intravaginal dose with samples collected at various times following dosing. Blood was collected by cardiac puncture. Blood was allowed to clot briefly at room temperature and serum was separated from the clot via centrifugation. The serum was stored at −20° C. until it was analyzed for cytokine concentrations.

Following blood collection, the rats were euthanized and their vaginal tract, including the cervix, was then removed and the tissue was weighed, placed in a sealed 1.8 mL cryovial and flash frozen in liquid nitrogen. The frozen vaginal tissue sample was then suspended in 1.0 mL of RPMI medium (Celox, St. Paul, Minn.) containing 10% fetal bovine serum (Atlas, Fort Collins, Colo.), 2 mM L-glutamine, penicillin/streptomycin and 2-mercaptoethanol (RPMI complete) combined with a protease inhibitor cocktail set III (Calbiochem, San Diego, Calif.). The tissue was homogenized using a Tissue Tearor (Biospec Products, Bartlesville, Okla.) for approximately one minute. The tissue suspension was then centrifuged at 2000 rpm for 10 minutes under refrigeration to pellet the debris, and the supernatant collected and stored at −20° C. until analyzed for cytokine concentrations.

ELISA kits for rat tumor necrosis factor-alpha (TNF) were purchased from BD PharMingen (San Diego, Calif.) and the rat monocyte chemoattractant protein-1 (MCP-1) ELISA kits were purchased from BioSource Intl. (Camarillo, Calif.). Both kits were performed according to manufacturer's specifications. Results for both TNF and MCP-1 are expressed in pg/mL and are normalized per 200 mg of tissue. The sensitivity of the TNF ELISA, based on the lowest value used to form the standard curve, is 63 pg/mL and for the MCP-1 ELISA it is 12 pg/mL.

### Viscosity Test Method

In the Examples below the viscosity is determined at 20±0.5° C. using a Haake RS series rheometer equipped with a 35 mm 2° cone using a controlled rate step test between 1 and 80 s⁻¹ with an interpolation at 16 s⁻¹ for viscosity versus shear rate. The values reported in the Examples are the values at 16 s⁻¹.

### Examples 8-10

The gels shown in Table 3 below were prepared using the following method.

Step 1: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 2: Edetate disodium was dissolved in water. Carbomer 974P was dispersed in the solution.
Step 3: The solution from Step 1 was added to the dispersion from Step 2 while mixing. 20% Tromethamine solution was added to the mixture to adjust the pH.

TABLE 3

| | Gels (% w/w) | | |
|---|---|---|---|
| Ingredient | Ex 8 | Ex 9 | Ex 10 |
| Drug (ethanesulfonate monohydrate) | 0.0153 | 0.0469 | 0.153 |
| Carbomer 974P | 1.50 | 1.50 | 1.70 |
| Propylene glycol | 15.00 | 15.00 | 15.00 |
| Methylparaben | 0.15 | 0.15 | 0.15 |
| Propylparaben | 0.03 | 0.03 | 0.03 |
| Edetate disodium | 0.05 | 0.05 | 0.05 |
| 20% Tromethamine solution | 0.54 | 0.85 | 1.25 |
| Purified water | 82.7147 | 82.5231 | 81.667 |
| pH | 3.8 | 4.0 | 4.0 |
| Viscosity (cps) | 6000 | 7000 | 7000 |

### Examples 11-18

The gels of Examples 11, 12, and 14 were prepared using the general method of Examples 8-10. The gels of Examples 13, 15, 16, 17, and 18 were prepared using the following method.

Step 1: The parabens were dissolved in propylene glycol (approximately 66 wt-% of the total amount used to achieve the final wt-% in the gel). 1-(2-Methylpropyl)-1H-imidazo[4,5-c][1,5]naphthyridin-4-amine ethanesulfonate monohydrate was then dissolved in the solution.
Step 2: Edetate disodium was dissolved in water. The remainder of the propylene glycol was added. Carbomer 974P was added and mixing was continued until the carbomer was completely hydrated.
Step 3: The solution from Step 1 was added to the dispersion from Step 2 while mixing. After mixing was complete the pH was measured.

TABLE 4

| | Gels (% w/w) | | | | | | | |
|---|---|---|---|---|---|---|---|---|
| Ingredient | Ex 11 | Ex 12 | Ex 13 | Ex 14 | Ex 15 | Ex 16 | Ex 17 | Ex 18 |
| Drug | 0.153 | 0.153 | 0.153 | 0.459 | 0.459 | 0.765 | 1.531 | 4.593 |
| (ethanesulfonate | | | | | | | | |
| monohydrate) | | | | | | | | |
| Carbomer 974P | 2.10 | 2.10 | 2.10 | 2.00 | 2.10 | 2.10 | 2.50 | 2.30 |
| Propylene glycol | 15.00 | 30.00 | 60.00 | 30.00 | 60.00 | 60.00 | 60.00 | 60.00 |
| Methylparaben | 0.15 | 0.15 | 0.15 | 0.15 | 0.15 | 0.15 | 0.15 | 0.15 |
| Propylparaben | 0.03 | 0.03 | 0.03 | 0.03 | 0.03 | 0.03 | 0.03 | 0.03 |
| Edetate disodium | 0.05 | 0.05 | 0.05 | 0.05 | 0.05 | 0.05 | 0.05 | 0.05 |
| 20% | 0.27 | 0.27 | 0.00 | 0.35 | 0.00 | 0.00 | 0.00 | 0.00 |
| Tromethamine | | | | | | | | |
| Purified water | 82.25 | 67.25 | 37.52 | 66.96 | 37.21 | 36.91 | 35.74 | 32.88 |
| pH | 4.0 | 4.2 | 3.8 | 4.3 | 3.6 | 3.2 | 2.9 | 2.5 |

The gels of Examples 11-18 were found to induce cytokines following a single dose using the test method described above with the following exceptions: the tissue samples were centrifuged at 3000 rpm for 10 minutes, all samples were subject to a dilution factor of 1:2 for TNF and 1:4 for MCP-1, and the sensitivity of the TNF ELISA, based on the lowest value used to form the standard curve, was 31 pg/mL. In addition, the gels of Examples 15, 17, and 18 were found to induce cytokines following a single dose using the test method described above.

### Examples 19-23

The gels shown in Table 5 below were prepared using the following method.

Step 1: The parabens were dissolved in propylene glycol (approximately 66 wt-% of the total amount used to achieve the final wt-% in the gel). 1-(2-Methylpropyl)-1H-imidazo[4,5-c][1,5]naphthyridin-4-amine ethanesulfonate monohydrate was then dissolved in the solution.
Step 2: Edetate disodium was dissolved in water. The remainder of the propylene glycol was added. Carbomer 974P and xanthan gum, if used, were added sequentially and mixing was continued until the thickener(s) was completely hydrated.
Step 3: The solution from Step 1 was added to the dispersion from Step 2 while mixing. After mixing was complete the pH was measured.

TABLE 5

| | Gels (% w/w) | | | | |
|---|---|---|---|---|---|
| Ingredient | Ex 19 | Ex 20 | Ex 21 | Ex 22 | Ex 23 |
| Drug (ethanesulfonate | 1.531 | 1.531 | 1.531 | 1.531 | 1.531 |
| monohydrate) | | | | | |
| Carbomer 974P | 1.25 | 1.50 | 1.50 | 2.00 | 2.50 |
| Xanthan gum | 1.75 | 1.50 | 1.50 | 1.00 | 0.00 |
| Propylene glycol | 50.00 | 50.00 | 60.00 | 60.00 | 60.00 |
| Methylparaben | 0.15 | 0.15 | 0.15 | 0.15 | 0.15 |
| Propylparaben | 0.03 | 0.03 | 0.03 | 0.03 | 0.03 |
| Edetate disodium | 0.05 | 0.05 | 0.05 | 0.05 | 0.05 |
| Purified water | 45.24 | 45.24 | 35.24 | 35.24 | 35.74 |
| pH | 3.0 | 2.8 | 3.2 | 3.1 | 2.9 |

The gels of Examples 19-23 were found to induce cytokines following a single dose using the test method described above.

### Examples 24-27

The gels of Examples 24-26 were prepared using the general method of Examples 19-23. The gel of Example 27 was prepared using the general method of Examples 8-10 except that the tromethamine was omitted.

TABLE 6

| | Gels (% w/w) | | | |
|---|---|---|---|---|
| Ingredient | Ex 24 | Ex 25 | Ex 26 | Ex 27 |
| Drug (ethanesulfonate | 1.531 | 1.531 | 1.531 | 1.531 |
| monohydrate) | | | | |
| Carbomer 974P | 2.00 | 1.00 | 2.00 | 2.50 |
| Xanthan gum | 1.00 | 2.00 | 1.00 | 0.00 |
| Propylene glycol | 50.00 | 50.00 | 60.00 | 60.00 |
| Methylparaben | 0.15 | 0.15 | 0.15 | 0.15 |
| Propylparaben | 0.03 | 0.03 | 0.03 | 0.03 |
| Edetate disodium | 0.05 | 0.05 | 0.05 | 0.05 |
| 20% Tromethamine solution | 1.44 | 1.00 | 0.59 | 0.00 |
| Purified water | 43.80 | 44.24 | 34.65 | 35.74 |
| pH | 3.4 | 3.5 | 3.4 | 3.1 |
| Viscosity (cps) | 11000 | 7550 | ND | ND |

ND = not determined

The gels of Examples 24-27 were found to induce cytokines following a single dose using the test method described above.

### Examples 28-32

The gels of Examples 28-32 were prepared using the general method of Examples 8-10 except that both the carbomer and xanthan gum were added in Step 2.

TABLE 7

| | Gels (% w/w) | | | | |
|---|---|---|---|---|---|
| Ingredient | Ex 28 | Ex 29 | Ex 30 | Ex 31 | Ex 32 |
| Drug | 0.15 | 0.45 | 0.75 | 1.50 | 4.50 |
| (ethanesulfonate | | | | | |
| monohydrate) | | | | | |
| Carbomer 974P | 2.20 | 2.33 | 2.50 | 2.00 | 1.75 |
| Xanthan gum | 1.10 | 1.17 | 1.25 | 1.00 | 0.88 |
| Propylene glycol | 15.00 | 30.00 | 30.00 | 40.00 | 60.00 |
| Methylparaben | 0.15 | 0.15 | 0.15 | 0.15 | 0.15 |
| Propylparaben | 0.03 | 0.03 | 0.03 | 0.03 | 0.03 |
| Edetate disodium | 0.05 | 0.05 | 0.05 | 0.05 | 0.05 |
| 20% | 0.50 | 1.25 | 0.67 | 1.70 | 2.00 |
| Tromethamine | | | | | |
| solution | | | | | |
| Purified water | 80.82 | 64.57 | 64.60 | 53.57 | 30.64 |
| pH | 4.0 | 4.0 | 3.5 | 3.5 | 3.0 |
| Viscosity | 8900 | 11000 | 8600 | 12000 | 17000 |

### Example 33

The gel shown in Table 8 below was prepared using the following method.

Step 1: Edetate disodium was dissolved in water (approximately 99% of the total amount used to achieve the final wt % in the gel).
Step 2: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 3: Carbomer 974P and xanthan gum were added sequentially to the solution from Step 1 while mixing and mixing was continued until the thickeners were completely hydrated.
Step 4: The solution from step 2 was added to the dispersion from step 3 while mixing.
Step 5: Tromethamine was dissolved in water (20% by weight tromethamine) and the solution was added to the gel from step 4 while mixing. Mixing was continued until the gel was uniform. After mixing was complete the pH was measured.

TABLE 8

| Ingredient | Example 33 Gel (% w/w) |
|---|---|
| Drug (ethanesulfonate monohydrate) | 0.15 |
| Carbomer 974P | 2.20 |
| Xanthan gum | 1.10 |
| Propylene glycol | 15.00 |
| Methylparaben | 0.15 |
| Propylparaben | 0.03 |
| Edetate disodium | 0.05 |
| Tromethamine | 0.20 |
| Purified water | 81.12 |
| pH | 3.8 |
| Viscosity (cps) | 10700 |

### Example 34

The gel shown in Table 9 below was prepared using the following method.

Step 1: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 2: Edetate disodium was dissolved in water (approximately half of the total amount used to achieve the final wt % in the gel).
Step 3: Carbomer 974P, xanthan gum, the solution from step 2, and water (approximately half of the total amount used to achieve the final wt % in the gel) were added sequentially to the solution from Step 1 while mixing and mixing was continued until the thickeners were completely hydrated.
Step 4: Tromethamine was dissolved in water (20% by weight tromethamine) and the solution was added to the gel from step 3 while mixing. Mixing was continued until the gel was uniform. After mixing was complete the pH was measured.

TABLE 9

| Ingredient | Example 34 Gel (% w/w) |
|---|---|
| Drug (ethanesulfonate monohydrate) | 1.50 |
| Carbomer 974P | 2.00 |
| Xanthan gum | 1.00 |
| Propylene glycol | 40.00 |
| Methylparaben | 0.15 |
| Propylparaben | 0.03 |
| Edetate disodium | 0.05 |
| Tromethamine | 0.40 |
| Purified water | 54.87 |
| pH | 3.6 |
| Viscosity (cps) | 7800 |

### Example 35

The gel shown in Table 10 below was prepared using the following method.

Step 1: The parabens were dissolved in propylene glycol. 1-(2-Methylpropyl)-1H-imidazo[4,5-c][1,5]naphthyridin-4-amine ethanesulfonate monohydrate was added in 3 separate portions while mixing. Mixing was continued until the drug was completely dissolved.
Step 2: Edetate disodium was dissolved in water (approximately a third of the total amount used to achieve the final wt % in the gel).
Step 3: Carbomer 974P, xanthan gum, the solution from step 2, and water (approximately two thirds of the total amount used to achieve the final wt % in the gel) were added sequentially to the solution from Step 1 while mixing and mixing was continued until the thickeners were completely hydrated.
Step 4: Tromethamine was dissolved in water (20% by weight tromethamine) and the solution was added to the gel from step 3 while mixing. Mixing was continued until the gel was uniform. After mixing was complete the pH was measured.

TABLE 10

| Ingredient | Example 35 Gel (% w/w) |
|---|---|
| Drug (ethanesulfonate monohydrate) | 4.50 |
| Carbomer 974P | 1.75 |
| Xanthan gum | 0.88 |
| Propylene glycol | 50.00 |
| Methylparaben | 0.15 |
| Propylparaben | 0.03 |
| Edetate disodium | 0.05 |
| Tromethamine | 0.65 |
| Purified water | 41.99 |
| pH | 3.0 |
| Viscosity (cps) | 7100 |

Other useful formulations are disclosed in copending U.S. application 60/698,416.

### Example 36

### Preparation of 1-(2-Methylpropyl)-1H-imidazo[4,5-c][1,5]naphthyridin-4-amine ethanesulfonate monohydrate

### Part A

Under a nitrogen atmosphere, a suspension of 3-nitro-1,5-naphthyridin-4-ol (1.00 kg, 5.23 mol) in DMF (4.5 L) was cooled in an ice bath. Phosphorous oxychloride (882.5 g, 5.75 mol) was added slowly over a period of one hour while maintaining the temperature at 16° C. to 20° C. After the addition was complete, the reaction was stirred for three hours at a temperature of 20° C. to 24° C. and then added quickly to two portions of demineralized water (12.5 L each) at 20° C. to 24° C. During the addition, the temperature of the mixtures was allowed to reach 29.5° C. to 30.5° C. The resulting mixtures were cooled to a temperature of approximately 10° C. over a period of 60 minutes. A solid formed in each mixture and was isolated by filtration, and each solid was washed with demineralized water (2×2 L and 1×1 L) until the pH of the filtrate equaled the pH of demineralized water. The tan solid product, 4-chloro-3-nitro[1,5]naphthyridine, contained water and was used in Part B within one hour.

### Part B

Isobutylamine (784 g, 10.7 mol) was added to a suspension of the material from Part A in tetrahydrofuran (6 L) over a period of 45 minutes while maintaining a reaction temperature of 17° C. to 27° C. When the addition was 75% complete, yellow needles formed in the solution. After the addition was complete, the reaction was stirred for 30 minutes at a temperature of 21.5° C. to 22.5° C. and then added with stirring to two portions of demineralized water (12 L each). The resulting mixtures were stirred for 30 minutes. The solid formed in each mixture was isolated by filtration, and each solid was washed with demineralized water (2×2 L) until the pH of the filtrate equaled the pH of demineralized water. The solids were then dried overnight on the filter funnels to provide 1.225 kg of N⁴-(2-methylpropyl)-3-nitro[1,5]naphthyridin-4-amine as a yellow solid, which was combined with material from another run.

### Part C

A hydrogenation vessel was charged with a suspension of N₄-(2-methylpropyl)-3-nitro[1,5]naphthyridin-4-amine (0.300 kg, 1.22 mol) in toluene (5 L), and magnesium sulfate (50 g) was added followed by 5% platinum on carbon (15 g) wet with toluene (500 mL). The reaction mixture was placed under hydrogen pressure (3.4×10⁵ Pa, 50 psi) on a Parr shaker at room temperature for 20 hours and then filtered through a layer of CELITE filter agent. The filter cake was washed with toluene (500 mL), and the filtrate was concentrated under reduced pressure to a volume of 4.5 L to provide a solution of N⁴-(2-methylpropyl)[1,5]naphthyridine-3,4-diamine in toluene.

### Part D

The solution from Part C was combined with p-toluenesulfonic acid monohydrate (11.4 g, 59.9 mmol). The reaction was heated to a temperature of 90° C., and triethyl orthoformate (0.178 kg, 1.2 mol) was added over a period of 60 minutes. After the addition was complete, the reaction mixture was heated at 100° C. for two hours, and the ethanol distillate (350 mL) was collected. The reaction mixture was cooled to room temperature, stirred overnight, and then treated with aqueous sodium carbonate (1 L of 1% w/w) and stored to provide 1-(2-methylpropyl)-1H-imidazo[4,5-c][1,5]naphthyridine in toluene and aqueous sodium carbonate. The material was combined with material from two additional runs on the same scale.

### Part E

The aqueous layer from the material from Part D was separated, and the toluene solution was washed with deionized water (2.7 L). The toluene layer was separated and concentrated under reduced pressure to a volume of 7 L. The toluene solution was then heated to 50° C., and peracetic acid (841 mL of 32% w/w in dilute acetic acid) was added over a period of two hours while maintaining the reaction temperature at 45° C. to 55° C. After the addition was complete, the reaction was heated at 50° C. overnight and then stirred at 40° C. to 50° C. while sodium metabisulfite (137 g in 3.33 L of deionized water) was added over a period of ten minutes. Aqueous sodium hydroxide (0.576 L of 50% w/w in 3.564 L water) was added over a period of 30 minutes. The resulting mixture was stirred for 30 minutes at approximately 50° C. and then cooled to a temperature of approximately 10° C. for one hour. A yellow precipitate was present and was isolated by filtration, washed with deionized water (5 L), dried by suction overnight, and further dried in a vacuum oven at 40° C. to 50° C. for about 24 hours to provide 0.693 kg of 1-(2-methylpropyl)-5-oxido-1H-imidazo[4,5-c][1,5]naphthyridine as a light yellow solid.

### Part F

Concentrated aqueous ammonium hydroxide (660 mL) was added to a suspension of 1-(2-methylpropyl)-5-oxido-1H-imidazo[4,5-c][1,5]naphthyridine (0.520 kg, 1.93 mol) in methanol (4.7 L), and the reaction temperature was adjusted to 20° C. Benzenesulfonyl chloride (0.716 kg, 4.05 mol) was added slowly over a period of 75 minutes while maintaining the reaction temperature below 26° C. with external cooling. After the addition was complete, the reaction was stirred for two hours at approximately 25° C. Sodium hydroxide (154 g in 2 L of deionized water) was then added to the reaction mixture while maintaining the reaction temperature at approximately 25° C. with external cooling. The resulting mixture was cooled to a temperature less than 10° C. for two hours. A precipitate formed and was isolated by filtration and washed sequentially with 3:2 methanol/deionized water (2×500 mL) and deionized water (7.5 L) until the pH of the filtrate was neutral. The filter cake was dried overnight on the filter funnel to provide 0.426 kg of 1-(2-methylpropyl)-1H-imidazo[4,5-c][1,5]naphthyridin-4-amine as a light yellow crystalline solid.

### Part G

A suspension of 1-(2-methylpropyl)-1H-imidazo[4,5-c][1,5]naphthyridin-4-amine (0.300 kg, 1.24 mol) in isopropyl alcohol (10 L) was heated slowly, and water (23 mL, 1.28 mol) was added. Aqueous ethanesulfonic acid (214 g of 70% w/w, 1.36 mol ethanesulfonic acid, 3.6 mol water) was added over a period of ten minutes at 60° C., and all the solids dissolved. After the addition was complete, the reaction was allowed to heat to 82° C. The reaction was cooled from 80° C. to 50° C. over a period of three hours (0.17° C./min) and then allowed to cool to room temperature overnight with slow stirring. White crystals were present. MTBE (10 L) was added, and the mixture was cooled to approximately 5° C. and maintained for two hours. The crystals were collected by filtration, washed with MTBE (10 L), dried overnight on the filter funnel, and further dried in a vacuum oven at 35° C. for three days to provide 372 g of 1-(2-methylpropyl)-1H-imidazo[4,5-c][1,5]naphthyridin-4-amine ethanesulfonate monohydrate as white needles. This material was characterized by powder X-ray diffraction analysis, TGA, and FTIR spectroscopy, and the data were found to be consistent with those shown in FIGS. 2, 7, and 4, respectively.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A salt selected from the group consisting of 1-(2-methylpropyl)-1H-imidazo[4,5-c][1,5]naphthyridin-4-amine ethanesulfonate and 1-(2-methylpropyl)-1H-imidazo[4,5-c][1,5]naphthyridin-4-amine methanesulfonate, or a solvate or hydrate thereof.

2. The salt of claim 1, wherein the salt is 1-(2-methylpropyl)-1H-imidazo[4,5-c][1,5]naphthyridin-4-amine ethanesulfonate, or a solvate or hydrate thereof.

3. The salt of claim 1, wherein the salt is 1-(2-methylpropyl)-1H-imidazo[4,5-c][1,5]naphthyridin-4-amine methanesulfonate, or a solvate or hydrate thereof.

4. The salt of claim 1 in dissolved form.

5. The salt of claim 1 in solid form.

6. The salt of claim 5 in crystalline form.

7. The salt of claim 5 in amorphous form.

8. The salt of claim 5 in solvated form.

9. The salt of claim 5 in hydrate form.

10. The salt of claim 9, in the form of a monohydrate.

11. A pharmaceutical composition comprising a pharmaceutically acceptable carrier and an effective amount of the salt of any preceding claim.

12. The pharmaceutical composition of claim 11 wherein the pharmaceutically acceptable carrier comprises water.

13. A method of treating a neoplastic disease in an animal, the method comprising administering a therapeutically effective amount of a salt of any one of claims 1 through 10, or a pharmaceutical composition of claim 11 or claim 12 to the animal.

14. A method of treating a viral disease in an animal, the method comprising administering a therapeutically effective amount of a salt of any one of claims 1 through 10, or a pharmaceutical composition of claim 11 or claim 12 to the animal.

15. A method of inducing cytokine biosynthesis in an animal, the method comprising administering an effective amount of a salt of any one of claims 1 through 10, or a pharmaceutical composition of claim 11 or claim 12 to the animal.

16. The method of claim 13, wherein the neoplastic disease is located in the cervix.

17. The method of claim 14, wherein the viral disease comprises human papilloma virus located in the cervix.

18. A method for preparing 1-(2-methylpropyl)-1H-imidazo[4,5-c][1,5]naphthyridin-4-amine ethanesulfonate, the method comprising:
combining 1-(2-methylpropyl)-1H-imidazo[4,5-c][1,5]naphthyridin-4-amine free base with ethanesulfonic acid and a carrier to form a mixture, wherein the carrier comprises an organic liquid and optionally water; and
allowing the components of the mixture to react under sufficient conditions to form 1-(2-methylpropyl)-1H-imidazo[4,5-c][1,5]naphthyridin-4-amine ethanesulfonate.

19. The method of claim 18 further comprising heating the free base, ethanesulfonic acid, and/or the carrier prior to combining them, and/or heating the mixture thereof.

20. The method of claim 18 or claim 19 further comprising forming a precipitate of the 1-(2-methylpropyl)-1H-imidazo[4,5-c][1,5]naphthyridin-4-amine ethanesulfonate in the mixture.

21. The method of claim 20 wherein forming a precipitate comprises cooling the mixture to form a precipitate.

22. The method of claim 21 wherein cooling occurs at a rate less than 2.0° C. per minute.

23. The method of any one of claims 18 through 22 wherein the carrier comprises at least two moles of water per mole of 1-(2-methylpropyl)-1H-imidazo[4,5-c][1,5]naphthyridin-4-amine free base present.

24. The method of claim 20 further comprising:
optionally adding an additional organic liquid to the mixture comprising the precipitate;
separating at least a portion of the precipitate from at least a portion of the mixture;
washing the precipitate; and
at least partially drying the precipitate.

25. The salt of claim 3 in crystalline form and having an X-ray powder diffraction pattern with peaks at 8.51 degrees two-theta, 14.12 degrees two-theta, 16.80 degrees two-theta, 17.88 degrees two-theta, 21.43 degrees two-theta, 23.24 degrees two-theta, and 29.16 degrees two-theta, wherein each of these values is ±0.15 degree two-theta.

26. The salt of claim 25 having an X-ray powder diffraction pattern with peaks at 7.15 degrees two-theta, 8.51 degrees two-theta, 14.12 degrees two-theta, 16.80 degrees two-theta, 17.88 degrees two-theta, 18.49 degrees two-theta, 18.88 degrees two-theta, 21.04 degrees two-theta, 21.43 degrees two-theta, 23.24 degrees two-theta, 25.40 degrees two-theta, 27.92 degrees two-theta, 28.77 degrees two-theta, and 29.16 degrees two-theta, wherein each of these values is ±0.15 degree two-theta.

27. The salt of claim 26 having an X-ray powder diffraction pattern with peaks at 7.15 degrees two-theta, 7.55 degrees two-theta, 8.51 degrees two-theta, 11.83 degrees two-theta, 13.65 degrees two-theta, 14.12 degrees two-theta, 14.87 degrees two-theta, 16.80 degrees two-theta, 17.88 degrees two-theta, 18.49 degrees two-theta, 18.88 degrees two-theta, 19.92 degrees two-theta, 20.24 degrees two-theta, 21.04 degrees two-theta, 21.43 degrees two-theta, 22.24 degrees two-theta, 23.24 degrees two-theta, 24.64 degrees two-theta, 25.40 degrees two-theta, 25.71 degrees two-theta, 27.20 degrees two-theta, 27.92 degrees two-theta, 28.77 degrees two-theta, 29.16 degrees two-theta, 30.94 degrees two-theta, 31.29 degrees two-theta, 32.76 degrees two-theta, 33.56 degrees two-theta, 34.04 degrees two-theta, 34.88 degrees two-theta, and 35.40 degrees two-theta, wherein each of these values is ±0.15 degree two-theta.

28. The salt of claim 3 having a unit cell with the following crystal interplanar spacings: about 10.38 Angstroms, about 6.27 Angstroms, about 5.27 Angstroms, about 4.96 Angstroms, about 4.14 Angstroms, about 3.82 Angstroms, and about 3.06 Angstroms.

29. The salt of claim 3 characterized by:
having an X-ray powder diffraction pattern with peaks at 7.15 degrees two-theta, 8.51 degrees two-theta, 14.12 degrees two-theta, 16.80 degrees two-theta, 17.88 degrees two-theta, 18.49 degrees two-theta, 18.88 degrees two-theta, 21.04 degrees two-theta, 21.43 degrees two-theta, 23.24 degrees two-theta, 25.40 degrees two-theta, 27.92 degrees two-theta, 28.77 degrees two-theta, and 29.16 degrees two-theta, wherein each of these values is +0.15 degree two-theta; and
a weight loss of 4.5% to 5.5% over a temperature range of 60° C. to 80° C. as measured by thermogravimetric analysis.

30. 1-(2-Methylpropyl)-1H-imidazo[4,5-c][1,5]naphthyridin-4-amine methanesulfonate having an X-ray powder diffraction pattern substantially as depicted in FIG. 1.

31. A method for preparing 1-(2-methylpropyl)-1H-imidazo[4,5-c][1,5]naphthyridin-4-amine methanesulfonate, the method comprising:
combining 1-(2-methylpropyl)-1H-imidazo[4,5-c][1,5]naphthyridin-4-amine free base with methanesulfonic acid and a carrier to form a mixture, wherein the carrier comprises an organic liquid and optionally water; and
allowing the components of the mixture to react under sufficient conditions to form 1-(2-methylpropyl)-1H-imidazo[4,5-c][1,5]naphthyridin-4-amine methanesulfonate.

32. The method of claim 31 further comprising heating the free base, methanesulfonic acid, and/or the carrier prior to combining them, and/or heating the mixture thereof.

33. The method of claim 31 or claim 32 further comprising forming a precipitate of the 1-(2-methylpropyl)-1H-imidazo[4,5-c][1,5]naphthyridin-4-amine methanesulfonate in the mixture.

34. The method of claim 33 wherein forming a precipitate comprises cooling the mixture to form a precipitate.

35. The method of any one of claims 31 through 34 wherein the carrier comprises at least two moles of water per mole of 1-(2-methylpropyl)-1H-imidazo[4,5-c][1,5]naphthyridin-4-amine free base present.

36. The method of claim 33 further comprising:
optionally adding an additional organic liquid to the mixture comprising the precipitate;
separating at least a portion of the precipitate from at least a portion of the mixture;
washing the precipitate; and
at least partially drying the precipitate.

37. A method of treating high risk cervical HPV infection by applying to the cervix a topical formulation comprising dissolved salt 1-(2-methylpropyl)-1H-imidazo[4,5-c][1,5]naphthyridin-4-amine methanesulfonate or 1-(2-methylpropyl)-1H-imidazo[4,5-c][1,5]naphthyridin-4-amine ethanesulfonate.
